(19) United States
(12) Patent Application Publication
(10) Pub. No.: US 2020/0072847 A1
(43) Pub. Date: Mar. 5, 2020

(54) SYSTEM AND METHODS FOR DIAGNOSING ACUTE INTERSTITIAL NEPHRITIS

(71) Applicants: Chirag Parikh, Lutherville-Timonium, MD (US); Dennis Moledina, New Haven, CT (US)

(72) Inventors: Chirag Parikh, Lutherville-Timonium, MD (US); Dennis Moledina, New Haven, CT (US)

(21) Appl. No.: 16/536,718

(22) Filed: Aug. 9, 2019

Related U.S. Application Data
(60) Provisional application No. 62/716,465, filed on Aug. 9, 2018.

(51) Int. Cl. G01N 33/68

(52) U.S. Cl. CPC G01N 33/6863; G01N 2333/715; G01N 2800/347; G01N 33/6893

(57) ABSTRACT

The invention provides methods and systems for detecting a biomarker related to AIN in a biological sample, and use thereof alone or as part of a diagnostic index for identifying and treating subjects at risk of AIN.

### CROSS REFERENCE TO RELATED APPLICATIONS

This application claims priority to U.S. Provisional Application No. 62/716,465, filed Aug. 9, 2018 which is hereby incorporated by reference herein in its entirety.

### STATEMENT REGARDING FEDERALLY SPONSORED RESEARCH OR DEVELOPMENT

This invention was made with government support under DK090203 and under K23DK117065 awarded by the National Institutes of Health (NIH). The government has certain rights in the invention.

### BACKGROUND OF THE INVENTION

Acute interstitial nephritis (AIN) is a common, preventable, and treatable cause of kidney disease. AIN is a form of immune-mediated kidney injury that can be triggered by use of medications such as antibiotics, proton pump inhibitors, and cancer immunotherapy agents (Moledina and Perazella, 2016, J Nephrol, 29(5):611-616; Nochaiwong et al., 2018, Nephrol Dial Transplant, 33(2):331-342). Ongoing inflammation in AIN leads to fibrosis and permanent kidney damage, and 40-60% of patients develop chronic kidney disease (CKD) after an episode of AIN (Muriithi et al., 2014, Am J Kidney Dis, 64(4):558-566; Simpson et al, 2006, Nephrology (Carlton), 11(5):381-385). An estimated 19,500 to 39,000 new cases of AIN occur in the U.S. each year from proton pump inhibitor use alone (Nochaiwong et al., 2018, Nephrol Dial Transplant, 33(2):331-342; Antoniou et al., 2015, CMAJ Open, 3(2):E166-171.4571830; Valluri et al., 2015, QJM, 108(7):527-532). A meta-analysis of nine studies found that long-term proton pump inhibitor (PPI) use was associated with a 36% higher risk of CKD and a 42% higher risk of end-stage renal disease, presumably from unrecognized AIN (Lazarus et al., 2016, JAMA Intern Med, 176(2):238-246; Xie et al., 2016, J Am Soc Nephrol, 27(10):3153-3163; Arora et al., 2016, BMC Nephrol, 17(1):112; Peng et al., 2016, Medicine (Baltimore), 95(15):e3363; Moledina and Perazella, 2016, J Am Soc Nephrol, 27(10):2926-2928). It is estimated that 2-5% of prevalent CKD cases are attributable to PPI use, equivalent to 0.5-1 million cases in the U.S. (Nochaiwong et al., 2018, Nephrol Dial Transplant, 33(2):331-342).

Kidney damage from AIN is reversible if it is recognized early, the offending drug is discontinued and immunosuppressive therapy is begun. However, the diagnosis of AIN is challenging because the symptoms and signs are all non-specific (Moledina and Perazella, 2016, J Nephrol, 29(5):611-616; Perazella, 2014, Clin Nephrol, 81(6):381-388). Clinically, cases with AIN are often overlooked because the loss in renal function occurs gradually over weeks to months (Chu et al., 2014, Clin J Am Soc Nephrol, 9(7): 1175-1182). Moreover, the current diagnostic tests for AIN, including urine eosinophils, urine sediment examination for leukocytes and leukocyte casts, and imaging tests, have poor sensitivity and specificity (Fogazzi et al., 2012, Am J Kidney Dis, 60(2):330-332; Muriithi et al., 2013, Clin J Am Soc Nephrol; 8(11):1857-1862; Perazella and Bomback, 2013, Clin J Am Soc Nephrol, 8(11):1841-1843). Thus, the diagnosis of AIN currently relies entirely on maintaining a high index of clinical suspicion for this disease and requires confirmation by a kidney biopsy.

Due to a 1-2% risk of severe bleeding with kidney biopsy, this procedure is often delayed due to comorbidities or concomitant medications that increase risk of bleeding, or not performed due to unacceptable risk (Corapi et al., 2012, Am J Kidney Dis, 60(1):62-73). AIN is suspected clinically in someone with acute to subacute loss of renal function by presence of subtle abnormalities on urine sediment examination and by exclusion of other causes of loss of renal function. These clinical clues were evaluated in isolation and showed poor accuracy (Perazella, 2014, Clin Nephrol, 81(6):381-388; Fogazzi et al., 2012, Am J Kidney Dis, 60(2):330-332; Muriithi et al., 2013, Clin J Am Soc Nephrol, 8(11):1857-1862).

Appropriately-designed, biopsy-based studies have led to biomarker discovery in various kidney diseases (Ju et al., 2015, Sci Transl Med, 7(316):316ra193; Baier and Hanson, 2004, Diabetes, 53(5): 1181-1186; Gohda et al., 2012, J Am Soc Nephrol, 23(3):516-524; Hayek et al., 2015, N Engl J Med, 373(20):1916-1925). However, past studies in AIN have failed to identify a diagnostic biomarker. These studies can be classified into three major types: (i) retrospective analysis of biopsy registries, which analyzed data that was generated for clinical use (Muriithi et al., 2014, Am J Kidney Dis, 64(4):558-566; Valluri et al., 2015, QJM, 108(7):527-532; Verde et al., 2012, Am J Nephrol, 35(3):230-237), (ii) studies that evaluated kidney tissue to describe cell-types involved in AIN (Zand et al., 2015, Clin Nephrol, 84(9):138-144; D'Agati et al., 1989, Mod Pathol, 2(4):390-396), and (iii) one published study that evaluated diagnostic biomarkers for AIN (Wu et al., 2010, Clinical Journal of the American Society of Nephrology, 5(11): 1954-1959), but each of these studies had several limitations. These limitations included that the registry studies did not collect biospecimens to identify biomarkers, the studies that evaluated kidney tissue did not include biomarker testing, and the study that did evaluate diagnostic biomarkers used healthy volunteers as controls, tested biomarkers of acute tubular injury (ATI), and used unadjudicated AIN biopsy reports as a gold-standard. While AIN can lead to ATI, the latter is often caused by other conditions such as sepsis, hypotension, and nephrotoxins, whose management differs from AIN.

Thus there is a need in the art for non-invasive diagnostic biomarkers of AIN and for systems and methods for using the biomarkers for determining appropriate treatment regimens. The current invention addresses these needs.

### SUMMARY OF THE INVENTION

In one embodiment, the invention relates to a system for detecting at least one marker associated with acute interstitial nephritis (AIN) in a biological sample from a subject. In one embodiment, the biological sample is a urine sample, a saliva sample, a mucous sample, a whole blood sample, a blood plasma sample, a semen sample or a milk sample obtained from the subject.

In one embodiment, at least one marker is a clinical marker or an inflammatory biomarker. In one embodiment, at least one marker is TNF-α, IL-9 or IL-5.

In one embodiment, the invention relates to the use of a system for detecting at least one marker associated with AIN in a biological sample from a subject for diagnosing an individual as having AIN or an increased risk of developing AIN.

In one embodiment, the invention relates to a method of diagnosing a subject as having AIN or an increased risk of developing AIN, comprising: detecting the level of at least one marker associated with AIN in a sample of the subject; comparing the level of the at least one marker to the level of the marker in a comparator control, and c) diagnosing the subject as having an increased risk of AIN based on detecting a significant difference between the level of the marker associated with AIN in the sample of the subject and the comparator control.

In one embodiment, the sample is a urine sample, a saliva sample, a mucous sample, a whole blood sample, a blood plasma sample, a semen sample or a milk sample obtained from the subject.

In one embodiment, at least one marker is a clinical marker or an inflammatory biomarker. In one embodiment, at least one biomarker is TNF-α, IL-9 or IL-5. In one embodiment, a risk of developing AIN is diagnosed when an increased level of at least one of TNF-α, IL-9 and IL-5 is detected as compared to a comparator control.

In one embodiment, the invention relates to a method of diagnosing a subject as having AIN or an increased risk of developing AIN, comprising the steps of: detecting the levels of at least two markers associated with AIN in at least one sample of a subject, determining a health profile of the subject based on the levels of the at least two markers associated with AIN, comparing the health profile of the subject to a diagnostic index generated from an analysis of AIN and non-AIN samples, and diagnosing the subject as having an increased risk of AIN based on the diagnostic index.

In one embodiment, the sample is a urine sample, a saliva sample, a mucous sample, a whole blood sample, a blood plasma sample, a semen sample or a milk sample obtained from the subject.

In one embodiment, at least one marker is a clinical marker or an inflammatory biomarker. In one embodiment, at least one marker is the level of blood eosinophils, the level of white blood cells in a urine sample, the level of hematuria, the level of albuminuria, the level of proteinuria, the baseline glomerular filtration rate, the level of TNF-α in a urine sample, the level of IL-5 in a urine sample, and the level of IL-9 in a urine sample.

In one embodiment, the invention relates to a method of treating a subject identified as having AIN or an increased risk of developing AIN, comprising the steps of: detecting the levels of at least two markers associated with AIN in at least one sample of a subject, determining a health profile of the subject based on the levels of the at least two markers associated with AIN, comparing the health profile of the subject to a diagnostic index generated from an analysis of AIN and non-AIN samples, diagnosing the subject as having an increased risk of AIN based on the diagnostic index, and administering a treatment regimen to the subject on the basis of the diagnosis.

In one embodiment, the sample is a urine sample, a saliva sample, a mucous sample, a whole blood sample, a blood plasma sample, a semen sample or a milk sample obtained from the subject.

In one embodiment, at least one marker is a clinical marker or an inflammatory biomarker. In one embodiment, at least one marker is the level of blood eosinophils, the level of white blood cells in a urine sample, the level of hematuria, the level of albuminuria, the level of proteinuria, the baseline glomerular filtration rate, the level of TNF-α in a urine sample, the level of IL-5 in a urine sample, and the level of IL-9 in a urine sample.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a STARD flow diagram of participant enrollment.

FIG. 2 depicts a comparison of urine TNF-α and IL-9 between AIN and controls in 2 subcohorts. Median (horizontal line), 25th and 75th percentiles (box), and 5th and 95th percentiles (whiskers) of biomarkers are shown. Biomarker values in pg/mg of creatinine. Wilcoxon's rank-sum test. Cohort 1 includes 22 participants with AIN and 105 without AIN; cohort 2 includes 10 participants with AIN and 81 without AIN. AIN, acute interstitial nephritis; Cr, creatinine. Values shown represent P values.

FIG. 3 depicts dot plots of biomarkers (on log scale). Wilcoxon Ranksum test. Cohort 1 includes 22 AIN participants and 105 without AIN; cohort 2 includes 10 AIN participants, and 81 without AIN. Line represents median value. AIN, acute interstitial nephritis; TNF, tumor necrosis factor; IL, interleukin, Cr, creatinine.

FIG. 4 depicts Urine TNF-α and IL-9 in participants with AIN compared with those with other kidney diseases and no kidney disease. Median (horizontal line), 25th and 75th percentiles (box), and 5th and 95th percentiles (whiskers) of biomarkers are shown. Both urine biomarkers are normalized to urine creatinine and shown in pg/mg. *0.001<P<0.05, and **P<0.001. Wilcoxon's rank-sum test comparing biomarker levels among AIN (n=32) and acute tubular necrosis (ATN; n=38), glomerular disease (GN; n=59), diabetic kidney disease (DKD; n=37), arterionephrosclerosis (fibrosis; n=24), other diagnoses (n=27), and participants without known kidney disease (n=20). Cr, creatinine.

FIG. 5 depicts the association of urine TNF-α and IL-9 with interstitial histological features. Median (horizontal line), 25th and 75th percentiles (box), and 5th and 95th percentiles (whiskers) of biomarkers. Both urine biomarkers are normalized to urine creatinine and shown in pg/mg. Wilcoxon's rank-sum test comparing biomarkers with interstitial histological features. Cr, creatinine. Values shown represent P values.

FIG. 6 depicts a table demonstrating the Association of urine biomarkers with AIN. Models 1 and 2 are univariable logistic regression analyses testing association of log-continuous biomarkers and quartiles of biomarkers with AIN. Model 3 is a multivariable logistic regression analysis testing association of both biomarkers with AIN (i.e., controlling for each other). Model 4 is a multivariable logistic regression analysis testing association of both biomarkers with AIN controlling for blood eosinophils and dipstick leukocytes and protein. AUC with 95% CI for model 1 was 0.76 (0.67, 0.85); model 2, 0.77 (0.68, 0.86); model 3, 0.79 (0.71, 0.88); and model 4, 0.84 (0.76, 0.91). All goodness-of-fit P values were greater than 0.05 (Hosmer-Lemeshow test). Q1-Q4 indicate quartiles of biomarkers and values in parentheses indicate quartile cutoffs in pg/mg of creatinine. AIN, acute interstitial nephritis; AUC, area under receiver operating characteristic curve.

FIG. 7A and FIG. 7B depict a comparison of AUC between clinicians, clinical model, and biomarkers. FIG. 7A depicts a comparison of AUC of clinical nephrologists' prebiopsy diagnosis with the model including biomarkers. FIG. 7B depicts a comparison of AUC of the clinical model consisting of blood eosinophils, and dipstick protein and white blood cells, with the model including biomarkers. P<0.001 for both comparisons of models with and without biomarkers (likelihood ratio test).

FIG. 8 depicts the association of clinicians' diagnosis, clinical tests, and biomarkers with AIN. Model 1 is a multivariable logistic regression analysis testing association of quartiles of biomarkers and clinicians' prebiopsy diagnosis with AIN. Model 2 is a multivariable logistic regression analysis testing association of blood eosinophils, dipstick leukocytes and protein, and quartiles of biomarkers with AIN. All goodness-of-fit P values were greater than 0.05 (Hosmer-Lemeshow test). Q1-Q4 indicate quartiles of biomarkers and values in parentheses indicate quartile cutoffs in pg/mg of creatinine. AIN, acute interstitial nephritis.

FIG. 9A through FIG. 9F depict post-test probabilities of AIN at a range of pretest probabilities at 2 cutoffs of IL-9. AUC for outcome of AIN versus all causes of AKD (FIG. 9A) and AIN versus ATI (FIG. 9B). Post-test probability of AIN at a range of pretest probabilities at IL-9 cutoff equal to median (FIG. 9C and FIG. 9D) and top 15% values (FIG. 9E and FIG. 9F). Top 15% cutoff was chosen based on 15% prevalence of AIN in cohort.

FIG. 10 depicts a table of the post-test probabilities of acute interstitial nephritis at a range of pre-test probabilities.

FIG. 11A through FIG. 11F depict post-test probabilities of acute interstitial nephritis at a range of pre-test probabilities at two cut-offs of tumor necrosis factor-α. Area under receiver operating characteristics curve (AUC) for outcome of acute interstitial nephritis (AIN) vs. all causes of acute kidney disease (FIG. 11A) and AIN vs. acute tubular injury (FIG. 11B). Post-test probability of AIN at a range of pre-test probabilities at TNF-α cut-off equal to median (FIG. 11C and FIG. 11E) and top 15% values (FIG. 11D and FIG. 11F). Top 15% cut-off was chosen based on 15% prevalence of AIN in cohort.

FIG. 12A through FIG. 12C depict immunofluorescence of kidney tissue for TNF-α and FcεRI. FIG. 12A depicts the median (horizontal line), 25th and 75th percentiles (box), and 5th and 95th percentiles (whiskers) of number of cells per low-power field by diagnosis. P value obtained using 2-tailed t test comparing cells per low-power field by diagnosis. FIG. 12B depicts representative images of immunostaining from AIN (top row) and not AIN (bottom row) samples immunostained for TNF-α (left column) or FcεRI (right column). TNF-α+ cells are noted by arrows. FcεRI+ mononuclear cells are shown by arrowheads. FIG. 12C depicts a scatter plot showing correlation of cells staining positive for TNF-α and FcεRI. Best fit line is shown.

FIG. 13 depicts the colocalization of TNF-α and FCER1 staining cells in patients with acute interstitial nephritis. Shown are high magnification (40×) representative images of 5 μm adjacent sections stained as follows: Right panel: FCERI staining and left Panel: TNFa staining. Arrow heads denote cells which stain positive for both markers. Boxed inset shows a group of TNFa-positive, FCERI-negative cells. G: glomerulus. Table denotes results of 101 cells counted from AIN participants in whom sections were aligned (n=4).

FIG. 14 depicts the association of eosinophil-related cytokine and chemokines in AIN. Median (horizontal line), 25th and 75th percentile (box), and 5th and 95th percentile (whiskers) of biomarkers are shown. Both urine biomarkers are normalized to urine creatinine and shown in pg/mg. Wilcoxon rank sum test comparing Eosinophilic AIN (n=16) to Non-eosinophilic AIN (n=16), non-eosinophilic AIN to non-AIN (n=24), and Kruskal Wallis test comparing biomarker levels between the three groups.

FIG. 15 depicts a summary of the cytokines and chemokines measured in the study and their general function.

FIG. 16 depicts the ordinal scale used by pathologists to record interstitial histologic features of acute interstitial nephritis.

FIG. 17 depicts the detection range and precision of biomarkers.

FIG. 18 depicts the effect of corticosteroid use on 6-month glomerular filtration rate by urine IL-9 and pre-biopsy kidney function. Linear regression model for outcome of 6 month eGFR and predictor as steroid use controlling for baseline GFR and albuminuria and includes interaction term steroid*biomarkers (Interaction P-value=0.02).

### DETAILED DESCRIPTION

The present invention relates to systems and methods for diagnosing AIN in a subject in need thereof. In one embodiment, the invention provides novel biomarkers associated with AIN. In another embodiment, the invention provides a diagnostic index for use in diagnosing a subject as having, or at risk of developing, AIN. In one embodiment, the invention relates to methods of preventing AIN through monitoring one or more biomarkers of AIN, or a diagnostic index, in a subject identified as having an increased risk of AIN. In one embodiment, the invention relates to methods of treating AIN in a subject in need thereof, including administering or altering a treatment regimen on the basis of one or more biomarkers of AIN, or a diagnostic index.

In one embodiment, the invention provides a method for diagnosing a subject as having, or at risk of developing, AIN including detecting the presence or absence of at least one AIN biomarker in a patient sample. The patient sample can be one or more of a urine sample, a saliva sample, a blood sample and a plasma sample. In one embodiment, the sample is from a patient who has been prescribed a therapeutic agent as part of a treatment regimen. In one embodiment, the sample is from a patient who has been prescribed a proton pump inhibitor (PPI). In one embodiment, the sample is from a patient who has been prescribed a proton pump inhibitor (PPI).

In one embodiment, the invention relates to a system that can be used for detecting AIN in a subject. In one embodiment, the invention provides a system for detection of AIN in a form of a point-of-care technology (POCT). In one embodiment, the invention provides a system for detecting AIN in a form of a hand held device. In one embodiment, a hand held device may interact with a POCT, such as a test strip. In one embodiment, a hand-held device may interface with a computer software, an application (app), or a web-based evaluation tool. In one embodiment, a computer software, app, or web-based evaluation tool can provide results to a physician (for example as part of an electronic medical record). In one embodiment, a handheld device interfacing with a computer software is useful for self-monitoring by an individual.

In another embodiment, the method of the invention may comprise any method known in the art to effectively detect a biomarker associated with AIN in a sample. Suitable methods include, but are not limited to, immunoassays, enzyme assays, mass spectrometry, biosensors, and chromatography. Thus, the method of the invention includes the use of any type of instrumentality to detect a biomarker associated with AIN.

The invention relates, in part, to the discovery that one or more biomarker associated with AIN is present in the urine of a patient who has AIN. Occurrence of an increased level of one or more of TNF-α, IL-9 and IL-5 in a patient's urine is an indicator that the patient has, or is at risk of developing, AIN. Thus, the invention can be used to assess the level of one or more of TNF-α, IL-9 and IL-5 in the urine of a subject at risk of AIN and administer or alter a treatment plan for the subject based on detection of an increased level of one or more of TNF-α, IL-9 and IL-5. Accordingly, the method of the invention provides a new and convenient platform for detecting AIN.

In some instances, the invention may take the form of a user-friendly point-of-use or point-of-care platform, for example a lateral flow device, having a sample application region and a readable detection region to indicate the presence or absence of one or more of TNF-α, IL-9 and IL-5 or variable levels of one or more of TNF-α, IL-9 and IL-5. In one embodiment, the readable detection region includes a test line and a control line, wherein the test line detects one or more of TNF-α, IL-9 and IL-5, and the control line detects the presence or absence of a marker present in the fluid being tested. Preferably, the fluid being tested is urine and the marker includes, but is not limited to IgG, IgD or IgA.

In one embodiment, the system of the invention detects the presence or absence of one or more of TNF-α, IL-9 and IL-5 or variable levels of one or more of TNF-α, IL-9 and IL-5 by way of a lateral flow immunoassay that utilizes strips of cellulose membrane onto which antibodies and other reagents are applied. For example, the test sample moves along the strip due to capillary action and reacts with the reagents at different points along the strip. The end result is the appearance or absence of a detectable line or spot.

In one embodiment, the lateral flow device can be in the form of a cartridge that can be read by a machine. Preferably, the machine is automated.

In one embodiment, the presence or absence of one or more of TNF-α, IL-9 and IL-5 or variable levels of one or more of TNF-α, IL-9 and IL-5 of the invention can be detected in a system that takes the form of a laboratory test, for example a type of numbered well plate (e.g., 96 well plate).

In one embodiment, the invention relates to a diagnostic index utilizing two or more of the markers associated with AIN described herein that increases the probability of distinguishing AIN from non-AIN subjects. In one embodiment, the diagnostic index includes determining the level of at least two clinical markers in a sample of a subject. Clinical markers that can be detected include, but are not limited to, markers of allergic reaction (e.g., blood eosinophil count), markers of renal inflammation (e.g., white blood cells on urine microscopy), the baseline glomerular filtration rate, and markers of glomerular disease (e.g., hematuria and albuminuria or proteinuria). In one embodiment, an increase in the level of at least one clinical marker is associated with AIN. In one embodiment, the clinical marker is a markers of allergic reaction (e.g., blood eosinophil count) or a marker of renal inflammation (e.g., white blood cells on urine microscopy). In one embodiment, a decrease in the level of at least one clinical marker is associated with AIN. In one embodiment, the clinical marker is a marker of glomerular disease (e.g., hematuria and albuminuria or proteinuria). In one embodiment, the clinical marker is the baseline glomerular filtration rate.

In one embodiment, the diagnostic index includes determining the level of at least one clinical marker in a sample of a subject and further determining the level of at least one inflammatory biomarker of AIN in a sample of a subject.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or in some instances ±10%, or in some instances ±5%, or in some instances ±1%, or in some instances ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, “affinity moiety” refers to a binding molecule, such as an antibody, aptamer, peptide or nucleic acid, that specifically binds to a particular target molecule to be detected in a testing sample.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “applicator,” as the term is used herein, is meant any device including, but not limited to, a hypodermic syringe, a pipette, an iontophoresis device, a patch, and the like, for administering the compositions of the invention to a subject.

The terms “biomarker” and “marker” are used herein interchangeably. They refer to a substance that is a distinctive indicator of a biological process, biological event and/or pathologic condition. A “marker,” as the term is used herein, refers to a molecule that can be detected. Therefore, a marker according to the present invention includes, but is not limited to, a nucleic acid, a polypeptide, a carbohydrate, a lipid, an inorganic molecule, an organic molecule, an analyte, a metabolite or a radiolabel, each of which may vary widely in size and properties. A “marker” can be detected using any means known in the art or by a previously unknown means that only becomes apparent upon consideration of the marker by the skilled artisan. A marker may be detected using a direct means, or by a method including multiple steps of intermediate processing and/or detection.

The phrase “biological sample” is used herein in its broadest sense. A sample may be of any biological tissue or fluid from which biomarkers of the present invention may be assayed. Examples of such samples include but are not limited to blood, lymph, urine, gynecological fluids, biopsies, amniotic fluid and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, and archival samples with known diagnosis, treatment and/or outcome history. Biological or body samples may also include sections of tissues such as frozen sections taken for histological purposes. The sample also encompasses any material derived by processing a biological or body sample. Derived materials include, but are not limited to, cells (or their progeny) isolated from the sample, proteins or nucleic acid molecules extracted from the sample. Processing of a biological or body sample may involve one or more of: filtration, distillation, extraction, concentration, inactivation of interfering components, addition of reagents, and the like.

As used herein, a “biosensor” is an analytical device for the detection of an analyte in a sample. Biosensors can comprise a recognition element, which can recognize or capture a specific analyte, and a transducer, which transmits the presence or absence of an analyte into a detectable signal.

As used herein, the term “data” generally refers to data reflective of the absolute and/or relative abundance (level) of a biomarker in a sample. As used herein, the term “dataset” refers to a set of data representing levels of each of one or more biomarkers of a panel of biomarkers in a reference population of subjects. A dataset can be used to generate a formula/classifier or diagnostic index of the invention. According to one embodiment, the dataset need not comprise data for each biomarker of the panel for each individual of the reference population. For example, the “dataset” when used in the context of a dataset to be applied to a formula can refer to data representing levels of each biomarker for each individual in one or more populations, but as would be understood can also refer to data representing levels of each biomarker for 99%, 95%, 90%, 85%, 80%, 75%, 70% or less of the individuals in each of said one or more populations and can still be useful for purposes of applying to a formula.

The term “comparator control,”, as used herein, relates to a level of expression or activity which may be determined at the same time as the test sample by using a sample previously collected and stored from a subject whose disease state, e.g. cancerous, non-cancerous, is/are known.

As used herein, the term “detection reagent” refers to an agent comprising an affinity moiety that specifically binds to a biomarker or other targeted molecule to be detected in a sample. Detection reagents may include, for example, a detectable moiety, such as a radioisotope, a fluorescent label, a magnetic label, and enzyme, or a chemical moiety such as biotin or digoxigenin. The detectable moiety can be detected directly, or indirectly, by the use of a labeled specific binding partner of the detectable moiety. Alternatively, the specific binding partner of the detectable moiety can be coupled to an enzymatic system that produces a detectable product.

As used herein, a “detector molecule” is a molecule that may be used to detect a compound of interest. Non-limiting examples of a detector molecule are molecules that bind specifically to a compound of interest, such as, but not limited to, an antibody, a cognate receptor, and a small molecule.

By the phrase “determining the level of marker concentration” is meant an assessment of the amount of a marker in a sample using technology available to the skilled artisan to detect a sufficient portion of any marker product.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold higher or more, as compared with a control.

“Differentially decreased expression” or “down regulation” refers to biomarker product levels which are at least 10% or more, for example, 20%, 30%, 40%, or 50%, 60%, 70%, 80%, 90% lower or less, and/or 0.9 fold, 0.8 fold, 0.6 fold, 0.4 fold, 0.2 fold, 0.1 fold or less, as compared with a control.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease, or disorder, the frequency with which such a sign or symptom is experienced by a patient, or both, is reduced.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

As used herein “endogenous” refers to any material from or produced inside the organism, cell, tissue or system.

As used herein, the term “exogenous” refers to any material introduced from or produced outside the organism, cell, tissue or system.

The term “expression” as used herein is defined as the transcription and/or translation of a particular nucleotide sequence driven by its promoter.

As used herein, an “immunoassay” refers to a biochemical test that measures the presence or concentration of a substance in a sample, such as a biological sample, using the reaction of an antibody to its cognate antigen, for example the specific binding of an antibody to a protein. Both the presence of the antigen or the amount of the antigen present can be measured.

As used herein, an “instructional material” includes a publication, a recording, a diagram, or any other medium of expression which can be used to communicate the usefulness of a component of the invention in a kit for detecting biomarkers disclosed herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The “level” of one or more biomarkers means the absolute or relative amount or concentration of the biomarker in the sample.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Polypeptide,” as used herein refers to a polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” or “peptide” as used herein are intended to encompass any amino acid sequence and include modified sequences such as glycoproteins. The term “polypeptide” or “protein” or “peptide” is specifically intended to cover naturally occurring proteins, as well as those which are recombinantly or synthetically produced. It should be noted that the term “polypeptide” or “protein” includes naturally occurring modified forms of the proteins, such as glycosylated forms.

As used herein, the term “providing a prognosis” refers to providing a prediction of the probable course and outcome of a disease, disorder or condition, including prediction of severity, duration, chances of recovery, etc. The methods can also be used to devise a suitable therapeutic plan, e.g., by indicating whether or not the condition is still at an early stage or if the condition has advanced to a stage where aggressive therapy would be ineffective.

“Sample”, “specimen” or “biological sample” as used herein means a biological material isolated from an individual. The biological sample may contain any biological material suitable for detecting the desired biomarkers, and may comprise cellular and/or non-cellular material obtained from the individual.

The term “solid support,” “support,” and “substrate” as used herein are used interchangeably and refer to a material or group of materials having a rigid or semi-rigid surface or surfaces. In one embodiment, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

By the term “specifically binds,” as used herein, is meant a molecule, such as an antibody, which recognizes and binds to another molecule or feature, but does not substantially recognize or bind other molecules or features in a sample.

The “therapeutic concentration” or “therapeutic level” is the concentration of a substance at which therapeutic benefits are gained.

The term “treatment regimen” or “medical regimen” as used herein relates to at least the frequency and dosage of any pharmaceutical agent being taken by an individual for treatment or prevention of a disease or condition.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the identification of diagnostic biomarkers of AIN and the development of diagnostic indices which were generated for biopsy-proven AIN. These diagnostic indices find use in research (e.g., to conduct clinical trials of drug withdrawal and/or immunosuppressive therapy in AIN) and in clinical practice to diagnose patients with suspected AIN without requiring a kidney biopsy.

The present invention relates to systems and methods for conveniently monitoring the presence or absence of at least one biomarker of AIN in a sample. In one embodiment, the sample is urine. Occurrence of increased levels of the biomarker of AIN in a patient's urine is an indicator that the patient has AIN. In one embodiment, the invention can be used to assess the risk of development of AIN. In one embodiment, the invention can be used to assess the risk of development of AIN in an individual who has been prescribed or administered another therapeutic agent (e.g, an antibiotic, a proton pump inhibitor or a chemotherapeutic agent.) Accordingly, the method of the invention provides a new and convenient platform for monitoring AIN risk in response to a particular treatment.

### Inflammatory Biomarkers

The present invention is based, in part, on the discovery that inflammatory biomarkers are present in urine samples and that an increased level of inflammatory biomarkers correlates with AIN. In one embodiment, the inflammatory biomarker is at least one of TNF-α, IL-5 and IL-9. Accordingly, the invention provides compositions and methods for detecting AIN from an analysis of a biological sample.

The detection and comparison of the levels of at least one of TNF-α, IL-5 and IL-9, in a biological sample can be both diagnostic and prognostic of AIN. For example, in one embodiment, an elevated level of at least one of TNF-α, IL-5 and IL-9 in a biological sample is indicative of AIN or of a greater risk or predisposition of the subject to develop AIN. Therefore, in various embodiments of the invention, the detection and measurement of the level of expression of at least one of TNF-α, IL-5 and 1-9 in a biological sample is used in a diagnostic assay, a prognostic assay, to monitor a clinical trial and in a screening assay.

In one embodiment, the invention provides diagnostic and prognostic assays for detecting at least one of TNF-α, IL-5 and IL-9.

In one embodiment, the invention relates to a method of detecting at least one of TNF-α, IL-5 and IL-9, in a biological sample, to determine the predisposition of a subject to develop AIN, to monitor the effect of a therapy administered to a subject, or to identify patients likely to respond to a therapy. In one embodiment, the method comprises: a) quantifying the level of at least one of TNF-α, IL-5 and IL-9, in a biological sample from a subject, and; b) comparing the level to that of a comparator control; wherein an increase in the level relative to that of the comparator control is indicative of AIN, i.e., it is an indication that the subject is suffering from AIN or has a predisposition to develop AIN. The level of at least one of TNF-α, IL-5 and IL-9, in a biological sample as compared to that of a comparator control can also be useful for monitoring the effect of a therapy administered to a subject. Further, the level of at least one of TNF-α, IL-5 and IL-9, in a biological sample as compared to that of a comparator control could identify patients who would respond to a specific treatment regimen (e.g., immunosuppressive therapy such as corticosteroids.)

In a particular embodiment, the biological sample is a urine sample, which can be obtained by conventional methods, e.g., by collection, by using methods well known to those of ordinary skill in the related medical arts. Samples can be obtained from subjects previously diagnosed or not with AIN.

Because of the variability of the diagnostic methods that may be used to detect a biomarker in a urine sample, the sample size required for analysis may range from 1 mL, 10 mL, 50 mL, 100 mL, 200 mL, 300 mL, or more than 500 mL. The appropriate sample size may be determined based on the method used to analyze the sample. The standard preparative steps for the determination are well known to one of ordinary skill in the art.

In a particular embodiment, with the aim of quantifying the level of at least one of TNF-α, IL-5 and IL-9, the method of the invention comprises (i) contacting the sample with a composition comprising one or more antibodies that specifically bind to one or more epitopes of at least one of TNF-α, IL-5 and 1-9 and (ii) quantifying the antibody-marker complexes that are formed. There is a wide range of immunological assays (immunoassays) available to detect and quantify the formation of specific antigen-antibody complexes; a number of protein-binding assays, competitive and non-competitive, have been previously described, and several of these are commercially available. Hence, the amount of at least one of TNF-α, IL-5 and 1-9 protein can be quantified by means of specific antibodies to at least one of TNF-α, IL-5 and 1-9. The antibodies can be in the form of monoclonal antibodies, polyclonal antibodies, intact or recombinant fragments of antibodies, combibodies and Fab or scFv of antibody fragments. These antibodies can be human, humanized or non-human in origin. The antibodies used in these assays can be labeled or unlabeled; the unlabeled antibodies can be used in agglutination assays; the labeled antibodies can be used in a wide range of assays. Antibody labels include radionucleotides, enzymes, fluorophores, chemiluminescent reagents, enzyme substrates or cofactors, enzyme inhibitors, particles, colorants and derivatives. There is a wide variety of assays well known to those skilled in the art that can be applied to the present invention, which use unlabeled antibodies as primary reagents and labeled antibodies as secondary reagents. These techniques include but are not limited to Western-blot or Western transfer, ELISA (Enzyme-linked immunosorbent assay), RIA (Radioimmunoassay), Competitive EIA (Competitive enzyme immunoassay), DAS-ELISA (Double antibody sandwich-ELISA), immunocyto-chemical and immunohistochemical techniques, techniques based on biochips or protein microarrays that use specific antibodies, and colloidal precipitation-based assays in formats such as dipsticks. Other techniques to detect and quantify at least one of TNF-α, IL-5 and IL-9 are affinity chromatography, ligand binding assays and lectin binding assays.

In some embodiments, the final step of the method of the invention involves comparing the level of at least one of TNF-α, IL-5 and 1-9 quantified in a biological sample obtained from the subject to the level of at least one of TNF-α, IL-5 and IL-9 in a comparator control sample (i.e., positive control, negative control, historical norm, baseline level or reference value). The level of at least one of TNF-α, IL-5 and IL-9 in comparator control samples can be determined by measuring the level of at least one of TNF-α, IL-5 and 1-9 in a urine sample from AIN-free subjects (i.e., negative control subjects with respect to AIN). An increase in the level of at least one of TNF-α, 1-5 and 1-9 in a biological sample from the subject under study relative to the level of at least one of TNF-α, 1-5 and 1-9 in a comparator control sample is indicative of AIN, i.e., it is an indication that said subject is suffering from AIN or has a predisposition to develop AIN. Further, the level of at least one of TNF-α, 1-5 and 1-9 in a biological sample as compared to that of a comparator control sample can be useful for monitoring the effect of the therapy administered to a subject (e.g., a subject who has been administered a pharmaceutical agent associated with a risk of AIN.)

In one embodiment, the method of the invention, based on the measurement of the level (concentration) of at least one of TNF-α, IL-5 and IL-9 in urine samples is highly sensitive and specific.

### Diagnostic Index

In one embodiment, the present invention relates to the identification of combinations of clinical factors and optionally one or more biomarkers of AIN to generate diagnostic indexes for diagnosing AIN or risk of AIN. Accordingly, the present invention features methods for identifying subjects who have or are at risk of developing AIN by detection of the factors and assessing the clinical factors disclosed herein. These factors, or otherwise health profile, are also useful for monitoring subjects undergoing treatments and therapies, and for selecting or modifying therapies and treatments to alternatives that would be efficacious in subjects determined by the methods of the invention to have AIN or an increased risk of developing AIN.

The present invention provides an index of for use in patient monitoring or diagnostics. An AIN index is calculated as a function of multiple markers, biomarkers or factors that strongly correlate to AIN. These factors may include clinical factors alone or a combination of clinical factors and AIN biomarkers.

The risk of developing AIN can be assessed by measuring one or more of the factors described herein, and comparing the presence and values of the factors to reference or index values. Such a comparison can be undertaken with mathematical algorithms or formula in order to combine information from results of multiple individual factors and other parameters into a single measurement or diagnostic index. Subjects identified as having AIN or an increased risk of AIN can optionally be selected to receive counseling, an increased frequency of monitoring, or treatment regimens, such as kidney biopsy or administration of alternative therapeutic compounds. For example, in one embodiment, a subject identified as having high urine IL-9 (high inflammation) and high baseline glomerular filtration rate may be administered a corticosteroid, whereas a subject identified as having high urine IL-9 (high inflammation) but a lower baseline glomerular filtration rate or subjects having a lower level of urine IL-9 may be administered a non-corticosteroid treatment.

The factors of the present invention can thus be used to generate a health profile or signature of subjects: (i) who do not have and are not expected to develop AIN and/or (ii) who have or expected to develop AIN. The health profile of a subject can be compared to a predetermined or reference profile to diagnose or identify subjects at risk for developing AIN, to monitor the response to a therapeutic treatment (e.g. an antibiotic, a proton pump inhibitor or a chemotherapeutic agent), and to monitor the effectiveness of a treatment or preventative measure for AIN. Data concerning the factors of the present invention can also be combined or correlated with other data or test results, such as, without limitation, measurements of clinical parameters or other algorithms for AIN or AIN-associated diseases.

In one embodiment the diagnostic index for diagnosing AIN is provided which integrates results from two or more tests for diagnosing AIN thereby providing a scoring system to be used in distinguishing AIN from non-AIN. Examples of the diagnostic tests that may be integrated to generate the diagnostic index include, but are not limited to, detecting the level of blood eosinophils, detecting the level of white blood cells in a urine sample, detecting the level of hematuria, detecting the level of albuminuria or proteinuria, detecting the glomerular filtration rate or detecting the level of an inflammatory biomarker of AIN. In one embodiment, at least two diagnostic tests are used in generating the index. The two or more diagnostic tests used in generating the index can diagnose AIN based on identification of changes in the same or different directions in a test sample relative to a comparator control. For example, in one embodiment, two or more diagnostic tests both assess an increase in the detected marker as compared to a comparator control (e.g., an increase in blood eosinophil count, an increase in white blood cells on urine microscopy, or a high baseline glomerular filtration rate). In another embodiment, at least one diagnostic test detects an increase in the detected marker as compared to a comparator control and at least one diagnostic test detects a decrease in the detected marker as compared to a comparator control (e.g., a decrease in the level of hematuria, albuminuria or proteinuria).

In one embodiment, diagnostic index of the invention comprises a combination of at least four tests which are used to generate a scoring system for the index. A first test may assess the blood eosinophil count, a second test may assess the level of white blood cells in a urine sample, a third test may assess the level of hematuria and a fourth test may assess the level of albuminuria or proteinuria.

In one embodiment, diagnostic index of the invention comprises a combination of at least five tests which are used to generate a scoring system for the index. A first test may assess the blood eosinophil count, a second test may assess the level of white blood cells in a urine sample, a third test may assess the level of hematuria, a fourth test may assess the level of albuminuria or proteinuria, and a fifth test may assess the level of at least one inflammatory biomarker associated with AIN. In one embodiment, at least one inflammatory biomarker associated with AIN is TNF-α or IL-9. In one embodiment, at least one inflammatory biomarker associated with AIN is TNF-α or IL-9. In one embodiment, the levels of both TNF-α and IL-9 are detected in a urine sample as part of the diagnostic index.

In one embodiment, the diagnostic index includes at least one additional factor. Exemplary additional factors that can be included in the diagnostic index include, but are not limited to, age, sex, race, family history of AIN and previous history of AIN. In one embodiment, an additional factor that is included in the diagnostic index is female sex.

One of skill in the art recognizes that for an individual test statistical analysis can be performed on a reference or normative population sample of cells to determine confidence levels of having AIN based on the results of that test. Accordingly for each test, a scale can be arbitrarily partitioned into regions having scores such that a correct combination of the scores provides a diagnostic index having a certain degree of confidence. The partitioning can be performed by conventional classification methodology including, but not limited to, histogram analysis, multivariable regression or other typical analysis or classification techniques. For example, one skilled in the art recognizes that multi-variable regression analysis may be performed to generate this partitioning or to analyze empirical/arbitrary partitioning in order to determine whether the composite clinical index has a higher degree of significance than each of the individual indices from respective tests.

Information obtained from the methods of the invention described herein can be used alone, or in combination with other information (e.g., age, race, sexual orientation, vital signs, blood chemistry, etc.) from the subject or from a biological sample obtained from the subject.

Various embodiments of the present invention describe mechanisms configured to monitor, track, and report levels of at least one clinical factor and optionally one or more biomarkers of AIN for use in generating a diagnostic index of an individual at multiple time points. In one embodiment, the system allows for the collection of data from multiple samples from an individual. The system can notify the user/evaluator about the likelihood of risk of developing AIN when a change (i.e. increase or decrease) in the diagnostic is detected in subsequent samples from a single individual. For example, in some implementations, the system records the diagnostic index entered into the system by the user/evaluator or automatically recorded by the system at various timepoints during a treatment regimen and applies algorithms to recognize patterns that predict whether the individual is at high risk of developing AIN in the absence of intervening treatment. The algorithmic analysis, for example, may be conducted in a central (e.g., cloud-based) system. Data uploaded to the cloud can be archived and collected, such that learning algorithms refine analysis based upon the collective data set of all patients. In some implementations, the system combines quantified clinical features and physiology to aid in diagnosing risk objectively, early, and at least semi-automatically based upon collected data.

In some embodiments, the system is for personal use and tracking by the individual subject. In some embodiment, the data from the system is uploaded to a central system and a provider evaluates the data and makes a diagnosis or recommendation. Providers, in some implementations, may perform a live analysis through real-time data feed between a POCT system and a remote evaluator computing system.

The system has several advantages. The system can be in a form of a kit or an application in the context of an electronic device, such as an electronic hand held device or even a wearable data collection device for convenience.

In some implementations, the system is used to track an individual's ongoing risk. To enable such ongoing assessment, in some embodiments, applications for assessment may be made available for download to or streaming on a wearable data collection device via a network-accessible content store or other content repositories, or other content collections. Content can range in nature from simple text, images, or video content or the like, to fully elaborated software applications (“apps”) or app suites. Content can be freely available or subscription based. Content can be stand-alone, can be playable on a wearable data-collection device based on its existing capabilities to play content (such as in-built ability to display text, images, videos, apps, etc., and to collect data), or can be played or deployed within a content-enabling framework or platform application that is designed to incorporate content from content providers. Content consumers can include individuals at risk of developing AIN as well as clinicians, physicians, research subjects and/or educators who wish to incorporate system modules into their professional practices.

In one embodiment, the system for assessing the risk of developing AIN of the invention can be implemented on a cell phone, tablet computer, a desk top computer, and the likes.

In one embodiment, the system of the invention can be in a medium that operates automatically behind the scenes in an electronic medical records database/software so that a notice automatically occurs if the data is designated to prompt an alert.

In another embodiment, the system of the invention can be in a format that encompasses “machine learning” so the process and comparator are update and improved as more information is entered and new analogs are developed.

### Assay Systems

In one embodiment, the invention provides methods and systems for detecting the presence or level of at least one biomarker of AIN in urine. In one embodiment, at least one biomarker of AIN is at least one of a clinical marker and an inflammatory biomarker of the invention. In one embodiment, at least one biomarker of AIN is TNF-α, IL-9 or IL-5. The at least one biomarker of AIN in urine may be identified by any suitable assay. A suitable assay may include one or more of an enzyme assay, an immunoassay, mass spectrometry, chromatography, electrophoresis, a biosensor, an antibody microarray, or any combination thereof. If an immunoassay is used it may be an enzyme-linked immunosorbant immunoassay (ELISA), a sandwich assay, a competitive assay, a radioimmunoassay (RIA), a lateral flow immunoassay, a Western Blot, an immunoassay using a biosensor, an immunoprecipitation assay, an agglutination assay, a turbidity assay or a nephelometric assay. In one embodiment, the method of detection is an immunoassay that utilizes a rapid immunoassay platform such as lateral flow.

Accordingly, the invention includes any platform for detecting at least one biomarker of AIN in a biological sample such as urine. In one embodiment, the system provides a convenient point-of-care device which can quickly detect the presence or absence of at least one biomarker of AIN in an at home or clinical setting. One non-limiting example of a point of care device is a lateral flow immunoassay. Lateral flow immunoassay utilizes strips of a membrane, preferably a cellulose membrane such as nitrocellulose, as the solid support for the immunoassay, onto which lines of reagent (e.g. antibody or antigen specific for the target analyte) can be applied. Multiple analytes can be assayed by spatially separating the location of the application areas of the reagents. Additional reagent pads can be used below the test line(s) for other critical reagents and sample conditioning materials. When sample is added to the test device, the solution will flow across the pads below the test lines and rehydrate the sample conditioning compound and the critical reagents for the assay and then pass across the specific test line and deposit a detection label which can be a visual indication (colloidal gold, colored latex or other labels known to those skilled in the art) or a label that requires an instrument to measure the signal (fluorescence, chemiluminesence). An additional material can be added above the test line to absorb fluid that passes by the test lines.

The end result is the appearance or absence of a colored line or spot, which can be compared to a control line. In some instances, the control line is useful for the detection of a marker of urine in order to ensure that the sample tested is indeed urine. Preferably, the marker of urine is present at a concentration significantly different in urine compared to the amount in other common matrices (i.e. blood) so as to validate that the sample tested is urine.

In one embodiment, the system may include a base or support layer and an absorbent matrix comprising at least one absorbent layer through which a liquid sample can flow along a flow path by force or by capillary action. The base layer may also be absorbent and be in fluid communication with the absorbent matrix, such that the flow path of liquid sample passes through both the absorbent matrix and the base layer. The flow path includes at least two regions, where the first region is a sample application region, and the second region is a detection region.

In one embodiment, immunoassays can be formatted in a sandwich format where two antibodies or binding partners specific for a molecule can be utilized to anchor and detect the analyte of interest. Smaller molecules can be detected using a competitive format where only one antibody or binding partner is utilized to detect the drug of interest. The assays can be formatted in a method that provides a positive read, in which a line appears when drug is present, or a negative read, in which the line disappears when the drug is present.

One embodiment of the invention involves the production of antibodies or binding partners with high specificity to the biomarker of interest for utilization in the immunoassay. The antibody should have high specificity to the target biomarker to permit the design of an immunoassay that allows monitoring of compliance of drug dosing. The production of the antibody will require the synthesis of a derivative that can be utilized to immunize animals. The derivative will be designed in a manner to maximize the recognition of the target molecule with minimal cross reactivity to other substances that may be present in the sample. The derivative is linked to a carrier protein to enhance the immune recognition and allow the production of antibodies. The antibodies can be polyclonal or more preferably monoclonal antibodies. The design and production of antibodies is well known to those skilled in the art. In one embodiment, the antibodies are antibodies against TNF-α, IL-9 or IL-5.

In one embodiment, at least one biomarker of AIN of the invention can be detected in a system that takes the form of a laboratory test, for example a type of numbered well plate (e.g., 96 well plate). In one embodiment, the lateral flow device can be in the form of a cartridge that can be read by a machine. Preferably, the machine is automated.

In one embodiment, the system of the invention includes (i) a POCT and (ii) a digital device. In one embodiment, a digital device interacts with a POCT. In one embodiment, a digital device analyzes the results from a POCT. In one embodiment, a digital device records the results from a POCT. In one embodiment, a digital device reports the results from a POCT. In one embodiment, a digital device analyzes, records and/or reports the results from multiple POCT.

The invention disclosed is not limited to the platform chosen to measure the presence or concentration of the at least one biomarker of AIN. Rapid tests are well known and can be formatted in a lateral flow, flow through, capillary, biosensor and a number of other formats.

Detecting an Analyte

The concentration of the analyte or biomarker in a sample may be determined by any suitable assay. A suitable assay may include one or more of the following methods, an enzyme assay, an immunoassay, mass spectrometry, chromatography, electrophoresis or an antibody microarray, or any combination thereof. Thus, as would be understood by one skilled in the art, the system and methods of the invention may include any method known in the art to detect a biomarker in a sample.

In one embodiment, the sample of the invention is a biological sample. The biological sample can originate from solid or fluid samples. Preferably the sample is a fluid sample. The sample of the invention may comprise urine, whole blood, blood serum, blood plasma, sweat, mucous, saliva, milk, semen and the like.

Immunoassays

In one embodiment, the systems and methods of the invention can be performed in the form of various immunoassay formats, which are well known in the art. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), fluorescence recovery/localization after photobleaching (FRAP/FLAP), a sandwich assay, a competitive assay, an immunoassay using a biosensor, an immunoprecipitation assay, an agglutination assay, a turbidity assay, a nephlelometric assay, etc.

In general, immunoassays involve contacting a sample suspected of containing a molecule of interest (such as the disclosed biomarker) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength.

There are two main types of immunoassays, homogeneous and heterogeneous. In homogeneous immunoassays, both the immunological reaction between an antigen and an antibody and the detection are carried out in a homogeneous reaction. Heterogeneous immunoassays include at least one separation step, which allows the differentiation of reaction products from unreacted reagents. A variety of immunoassays can be used to detect one or more of the proteins disclosed or incorporated by reference herein.

ELISA is a heterogeneous immunoassay, which can be used in the methods disclosed herein. The assay can be used to detect protein antigens in various formats. In the “sandwich” format the antigen being assayed is held between two different antibodies. In this method, a solid surface is first coated with a solid phase antibody. The test sample, containing the antigen (e.g., a diagnostic protein), or a composition containing the antigen, such as a urine sample from a subject of interest, is then added and the antigen is allowed to react with the bound antibody. Any unbound antigen is washed away. A known amount of enzyme-labeled antibody is then allowed to react with the bound antigen. Any excess unbound enzyme-linked antibody is washed away after the reaction. The substrate for the enzyme used in the assay is then added and the reaction between the substrate and the enzyme produces a color change. The amount of visual color change is a direct measurement of specific enzyme-conjugated bound antibody, and consequently the antigen present in the sample tested.

ELISA can also be used as a competitive assay. In the competitive assay format, the test specimen containing the antigen to be determined is mixed with a precise amount of enzyme-labeled antigen and both compete for binding to an anti-antigen antibody attached to a solid surface. Excess free enzyme-labeled antigen is washed off before the substrate for the enzyme is added. The amount of color intensity resulting from the enzyme-substrate interaction is a measure of the amount of antigen in the sample tested. A heterogeneous immunoassay, such as an ELISA, can be used to detect any of the proteins disclosed or incorporated by reference herein.

Homogeneous immunoassays include, for example, the Enzyme Multiplied Immunoassay Technique (EMIT), which typically includes a biological sample comprising the biomarkers to be measured, enzyme-labeled molecules of the biomarkers to be measured, specific antibody or antibodies binding the biomarkers to be measured, and a specific enzyme chromogenic substrate. In a typical EMIT, excess of specific antibodies is added to a biological sample. If the biological sample contains the proteins to be detected, such proteins bind to the antibodies. A measured amount of the corresponding enzyme-labeled proteins is then added to the mixture. Antibody binding sites not occupied by molecules of the protein in the sample are occupied with molecules of the added enzyme-labeled protein. As a result, enzyme activity is reduced because only free enzyme-labeled protein can act on the substrate. The amount of substrate converted from a colorless to a colored form determines the amount of free enzyme left in the mixture. A high concentration of the protein to be detected in the sample causes higher absorbance readings. Less protein in the sample results in less enzyme activity and consequently lower absorbance readings. Inactivation of the enzyme label when the antigen-enzyme complex is antibody-bound makes the EMIT a useful system, enabling the test to be performed without a separation of bound from unbound compounds as is necessary with other immunoassay methods. A homogenous immunoassay, such as an EMIT, can be used to detect any of the proteins disclosed or incorporated by reference herein.

In many immunoassays, as described elsewhere herein, detection of antigen is made with the use of antigens specific antibodies as detector molecules. However, immunoassays and the systems and methods of the present invention are not limited to the use of antibodies as detector molecules. Any substance that can bind or capture the antigen within a given sample may be used. Aside from antibodies, suitable substances that can also be used as detector molecules include but are not limited to enzymes, peptides, proteins, and nucleic acids. Further, there are many detection methods known in the art in which the captured antigen may be detected. In some assays, enzyme-linked antibodies produce a color change. In other assays, detection of the captured antigen is made through detecting fluorescent, luminescent, chemiluminescent, or radioactive signals. The system and methods of the current invention is not limited to the particular types of detectable signals produced in an immunoassay.

Immunoassay kits are also included in the invention. These kits include, in separate containers (a) monoclonal antibodies having binding specificity for the polypeptides used in the diagnosis of inflammation or the source of inflammation; and (b) and anti-antibody immunoglobulins. This immunoassay kit may be utilized for the practice of the various methods provided herein. The monoclonal antibodies and the anti-antibody immunoglobulins can be provided in an amount of about 0.001 mg to 100 grams, and more preferably about 0.01 mg to 1 gram. The anti-antibody immunoglobulin may be a polyclonal immunoglobulin, protein A or protein G or functional fragments thereof, which may be labeled prior to use by methods known in the art. In several embodiments, the immunoassay kit includes two, three or four of: antibodies that specifically bind a protein disclosed or incorporated herein.

In one embodiment, the immunoassay kit of the invention can comprise (a) a sample pad, (b) a conjugated label pad, the conjugated label pad having a detectable label, a portion of the conjugated label pad and a portion of the sample pad forming a first interface, (c) a lateral-flow assay comprising a membrane, a portion of the membrane and a portion of the conjugated label pad forming a second interface, and (d) at least one antibody bound to the membrane, the first interface allowing fluid to flow from the sample pad to the conjugated label pad and contact the detectable label wherein the biomarker present in the sample forms an biomarker-conjugated label complex, the second interface allowing fluid to flow from the conjugated label pad to the membrane and to contact the at least one membrane-bound antibody to form to an biomarker-antibody complex and cause the detectable label to form a detectable signal.

In one embodiment, the immunoassay kit of the invention includes an additional component including but not limited to one or more of instructional material and sample collection receptacles. In one embodiment, the kit of the invention includes a single immunoassay system. In one embodiment, the kit of the invention includes more than one immunoassay system.

In one embodiment, the kit of the invention includes a handheld device. In one embodiment, the kit includes a system for or access to a computer software for analyzing, recording, monitoring, tracking and/or reporting the results of the POCT of the invention.

Mass Spectrometry and Chromatography

In one embodiment, the method of detection is a lab based test. In one embodiment, the lab based test is a semi-quantitative liquid chromatography-tandem mass spectrometry (LC-MS/MS) urine assay.

In one embodiment, the systems and methods of the invention can be performed in the form of various mass spectrometry (MS) or chromatography formats, which are well known in the art. As such, the levels of biomarkers present in a sample can be determined by mass spectrometry. Generally, any mass spectrometric techniques that can obtain precise information on the mass of peptides, and preferably also on fragmentation and/or (partial) amino acid sequence of selected peptides, are useful herein. Suitable peptide MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein.

The terms “mass spectrometry” or “MS” as used herein refer to methods of filtering, detecting, and measuring ions based on their mass-to-charge ratio, or “m/z.” In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). For examples see U.S. Pat. Nos. 6,204,500, 6,107,623, 6,268,144, 6,124,137; Wright et al., 1999, Prostate Cancer and Prostatic Diseases 2: 264-76; Merchant et al., 2000, Electrophoresis 21: 1164-67, each of which is hereby incorporated by reference in its entirety, including all tables, figures, and claims. Mass spectrometry methods are well known in the art and have been used to quantify and/or identify biomolecules, such as proteins and hormones (Li et al., 2000, Tibtech. 18:151-160; Starcevic et. al., 2003, J. Chromatography B, 792: 197-204; Kushnir et. al., 2006, Clin. Chem. 52:120-128; Rowley et al., 2000, Methods 20: 383-397; Kuster et al., 1998, Curr. Opin. Structural Biol. 8: 393-400). Further, mass spectrometric techniques have been developed that permit at least partial de novo sequencing of isolated proteins (Chait et al., 1993, Science, 262:89-92; Keough et al., 1999, Proc. Natl. Acad. Sci. USA. 96:7131-6; Bergman, 2000, EXS 88:133-44). Various methods of ionization are known in the art. For examples, Atmospheric Pressure Chemical Ionization (APCI) Chemical Ionization (CI) Electron Impact (EI) Electrospray Ionization (ESI) Fast Atom Bombardment (FAB) Field Desorption/Field Ionization (FD/FI) Matrix Assisted Laser Desorption Ionization (MALDI) and Thermospray Ionization (TSP).

The levels of biomarkers present in a sample can be determined by MS such as matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; tandem mass spectrometry (e.g., MS/MS, MS/MS/MS etc.); electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)ⁿ; atmospheric pressure photoionization mass spectrometry (APPI-MS); APPI-MS/MS; APPI-(MS)ⁿ; liquid chromatography-mass spectrometry (LC-MS), gas chromatography-mass spectrometry (GC-MS); high performance liquid chromatography-mass spectrometry (HPLC-MS); capillary electrophoresis-mass spectrometry; and nuclear magnetic resonance spectrometry. Peptide ion fragmentation in tandem MS (MS/MS) arrangements may be achieved using manners established in the art, such as, e.g., collision induced dissociation (CID). See for example, U.S. Patent Application Nos: 20030199001, 20030134304, 20030077616, which are herein incorporated by reference in their entirety. Such techniques may be used for relative and absolute quantification and also to assess the ratio of the biomarker according to the invention with other biomarkers that may be present. These methods are also suitable for clinical screening, prognosis, monitoring the results of therapy, identifying patients most likely to respond to a particular therapeutic treatment, for drug screening and development, and identification of new targets for drug treatment.

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. Nos. 5,118,937, and 5,045,694. In SELDI, the substrate surface is modified so that it is an active participant in the desorption process. In one variant, the surface is derivatized with adsorbent and/or capture reagents that selectively bind the biomarker of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. SELDI is a powerful tool for identifying a characteristic “fingerprint” of proteins and peptides in body fluids and tissues for a given condition, e.g. drug treatments and diseases. This technology utilizes protein chips to capture proteins/peptides and a time-of-flight mass spectrometer (tof-MS) to quantitate and calculate the mass of compounds ranging from small molecules and peptides of less than 1,000 Da up to proteins of 500 kDa. Quantifiable differences in protein/peptide patterns can be statistically evaluated using automated computer programs which represent each protein/peptide measured in the biofluid spectrum as a coordinate in multi-dimensional space. The SELDI system also has a capability of running hundreds of samples in a single experiment. In addition, all the signals from SELDI mass spectrometry are derived from native proteins/peptides (unlike some other proteomics technologies which require protease digestion), thus directly reflecting the underlying physiology of a given condition.

In MALDI and SELDI, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material. For additional information regarding mass spectrometers, see, e.g., Principles of Instrumental Analysis, 3rd edition, Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094. Detection and quantification of the biomarker will typically depend on the detection of signal intensity. For example, in certain embodiments, the signal strength of peak values from spectra of a first sample and a second sample can be compared (e.g., visually, by computer analysis etc.), to determine the relative amounts of particular biomarker. Software programs such as the Biomarker Wizard program (Ciphergen Biosystems, Inc., Fremont, Calif.) can be used to aid in analyzing mass spectra. The mass spectrometers and their techniques are well known to those of skill in the art.

In an embodiment, detection and quantification of biomarkers by mass spectrometry may involve multiple reaction monitoring (MRM), such as described among others by Kuhn et al. 2004 (Proteomics 4: 1175-86).

In an embodiment, MS peptide analysis methods may be advantageously combined with upstream peptide or protein separation or fractionation methods, such as for example with the chromatographic and other methods described herein below.

Chromatography can also be used for measuring biomarkers. As used herein, the term “chromatography” encompasses methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Chromatography as used herein may be preferably columnar (i.e., wherein the stationary phase is deposited or packed in a column), preferably liquid chromatography, and yet more preferably high-performance liquid chromatograph7 (HPLC). While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993.

Exemplary types of chromatography include, without limitation, HPLC, normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilized metal affinity chromatography, and the like.

In an embodiment, chromatography, including single-, two- or more-dimensional chromatography, may be used as a peptide fractionation method in conjunction with a further peptide analysis method, such as for example, with a downstream mass spectrometry analysis as described elsewhere in this specification.

Further peptide or polypeptide separation, identification or quantification methods may be used, optionally in conjunction with any of the above described analysis methods, for measuring at least one biomarker of the invention. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

Point-of-Use Devices

Point-of-use analytical tests have been developed for the routine identification or monitoring of health-related conditions (such as pregnancy, cancer, endocrine disorders, infectious diseases or drug abuse) using a variety of biological samples (such as urine, serum, plasma, blood, saliva). Some of the point-of-use assays are based on highly specific interactions between specific binding pairs, such as antigen/antibody, hapten/antibody, lectin/carbohydrate, apoprotein/cofactor and biotin/(strept)avidin. In some point-of use devices, assays are performed with test strips in which a specific binding pair member is attached to a mobilizable material (such as a metal sol or beads made of latex or glass) or an immobile substrate (such as glass fibers, cellulose strips or nitrocellulose membranes). Other point-of use devices may comprise optical biosensors, photometric biosensors, electrochemical biosensor, or other types of biosensors. Suitable biosensors in point-of-use devices for performing methods of the invention include “cards” or “chips” with optical or acoustic readers. Biosensors can be configured to allow the data collected to be electronically transmitted to the physician for interpretation and thus can form the basis for e-medicine, where diagnosis and monitoring can be done without the need for the patient to be in proximity to a physician or a clinic.

Detection of a biomarker in a sample can be carried out using a sample capture device, such as a lateral flow device (for example a lateral flow test strip) that allows detection of one or more biomarkers, such as those described herein.

The test strips of the present invention include a flow path from an upstream sample application area to a test site. For example, the flow path can be from a sample application area through a mobilization zone to a capture zone. The mobilization zone may contain a mobilizable marker that interacts with an analyte or analyte analog, and the capture zone contains a reagent that binds the analyte or analyte analog to detect the presence of an analyte in the sample.

Examples of migration assay devices, which usually incorporate within them reagents that have been attached to colored labels, thereby permitting visible detection of the assay results without addition of further substances are found, for example, in U.S. Pat. No. 4,770,853 (incorporated herein by reference). There are a number of commercially available lateral-flow type tests and patents disclosing methods for the detection of large analytes (MW greater than 1,000 Daltons) as the analyte flows through multiple zones on a test strip. Examples are found in U.S. Pat. Nos. 5,229,073, 5,591,645; 4,168,146; 4,366,241; 4,855,240; 4,861,711; 5,120,643 (each of which are herein incorporated by reference). Multiple zone lateral flow test strips are disclosed in U.S. Pat. Nos. 5,451,504, 5,451,507, and 5,798,273 (incorporated by reference herein). U.S. Pat. No. 6,656,744 (incorporated by reference) discloses a lateral flow test strip in which a label binds to an antibody through a streptavidin-biotin interaction.

Flow-through type assay devices were designed, in part, to obviate the need for incubation and washing steps associated with dipstick assays. Flow-through immunoassay devices involve a capture reagent (such as one or more antibodies) bound to a porous membrane or filter to which a liquid sample is added. As the liquid flows through the membrane, target analyte (such as protein) binds to the capture reagent. The addition of sample is followed by (or made concurrent with) addition of detector reagent, such as labeled antibody (e.g., gold-conjugated or colored latex particle-conjugated protein). Alternatively, the detector reagent may be placed on the membrane in a manner that permits the detector to mix with the sample and thereby label the analyte. The visual detection of detector reagent provides an indication of the presence of target analyte in the sample. Representative flow-through assay devices are described in U.S. Pat. Nos. 4,246,339; 4,277,560; 4,632,901; 4,812,293; 4,920,046; and 5,279,935; U.S. Patent Application Publication Nos. 20030049857 and 20040241876; and WO 08/030,546. Migration assay devices usually incorporate within them reagents that have been attached to colored labels, thereby permitting visible detection of the assay results without addition of further substances. See, for example, U.S. Pat. No. 4,770,853; PCT Publication No. WO 88/08534.

There are a number of commercially available lateral flow type tests and patents disclosing methods for the detection of large analytes (MW greater than 1,000 Daltons). U.S. Pat. No. 5,229,073 describes a semiquantitative competitive immunoassay lateral flow method for measuring plasma lipoprotein levels. This method utilizes a plurality of capture zones or lines containing immobilized antibodies to bind both the labeled and free lipoprotein to give a semi-quantitative result. In addition, U.S. Pat. No. 5,591,645 provides a chromatographic test strip with at least two portions. The first portion includes a movable tracer and the second portion includes an immobilized binder capable of binding to the analyte. Additional examples of lateral flow tests for large analytes are disclosed in the following patent documents: U.S. Pat. Nos. 4,168,146; 4,366,241; 4,855,240; 4,861,711; and 5,120,643; WO 97/06439; WO 98/36278; and WO 08/030,546.

Devices described herein generally include a strip of absorbent material (such as a microporous membrane), which, in some instances, can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the specific binding partner(s) and/or other reagents required for the detection and/or quantification of the particular analyte being tested for, for example, one or more proteins disclosed herein. Thus these zones can be viewed as functional sectors or functional regions within the test device.

In general, a fluid sample is introduced to the strip at the proximal end of the strip, for instance by dipping or spotting. A sample is collected or obtained using methods well known to those skilled in the art. The sample containing the particular proteins to be detected may be obtained from any biological source. In a particular example, the biological source is urine. The sample may be diluted, purified, concentrated, filtered, dissolved, suspended or otherwise manipulated prior to assay to optimize the immunoassay results. The fluid migrates distally through all the functional regions of the strip. The final distribution of the fluid in the individual functional regions depends on the adsorptive capacity and the dimensions of the materials used.

In some embodiments, porous solid supports, such as nitrocellulose, described elsewhere herein are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm).

Another common feature to be considered in the use of assay devices is a means to detect the formation of a complex between an analyte (such as one or more proteins described herein) and a capture reagent (such as one or more antibodies). A detector (also referred to as detector reagent) serves this purpose. A detector may be integrated into an assay device (for example includes in a conjugate pad), or may be applied to the device from an external source.

A detector may be a single reagent or a series of reagents that collectively serve the detection purpose. In some instances, a detector reagent is a labeled binding partner specific for the analyte (such as a gold-conjugated antibody for a particular protein of interest).

In other instances, a detector reagent collectively includes an unlabeled first binding partner specific for the analyte and a labeled second binding partner specific for the first binding partner and so forth. Thus, the detector can be a labeled antibody specific for a protein described herein. The detector can also be an unlabeled first antibody specific for the protein of interest and a labeled second antibody that specifically binds the unlabeled first antibody. In each instance, a detector reagent specifically detects bound analyte of an analyte-capture reagent complex and, therefore, a detector reagent preferably does not substantially bind to or react with the capture reagent or other components localized in the analyte capture area. Such non-specific binding or reaction of a detector may provide a false positive result. Optionally, a detector reagent can specifically recognize a positive control molecule (such as a non-specific human IgG for a labeled Protein A detector, or a labeled Protein G detector, or a labeled anti-human Ab(Fc)) that is present in a secondary capture area.

Flow-Through Device Construction and Design

A flow-through device involves a capture reagent (such as one or more antibodies) immobilized on a solid support, typically, microtiter plate or a membrane (such as, nitrocellulose, nylon, or PVDF). In a simple representative format, the membrane of a flow-through device is placed in functional or physical contact with an absorbent layer, which acts as a reservoir to draw a fluid sample through the membrane. Optionally, following immobilization of a capture reagent, any remaining protein-binding sites on the membrane can be blocked (either before or concurrent with sample administration) to minimize nonspecific interactions.

In operation of a flow-through device, a fluid sample is placed in contact with the membrane. Typically, a flow-through device also includes a sample application area (or reservoir) to receive and temporarily retain a fluid sample of a desired volume. The sample passes through the membrane matrix. In this process, an analyte in the sample (such as one or more protein, for example, one or more proteins described herein) can specifically bind to the immobilized capture reagent (such as one or more antibodies). Where detection of an analyte-capture reagent complex is desired, a detector reagent (such as labeled antibodies that specifically bind one or more proteins) can be added with the sample or a solution containing a detector reagent can be added subsequent to application of the sample. If an analyte is specifically bound by capture reagent, a characteristic attributable to the particular detector reagent can be observed on the surface of the membrane. Optional wash steps can be added at any time in the process, for instance, following application of the sample, and/or following application of a detector reagent.

Lateral Flow Device Construction and Design

Lateral flow devices are commonly known in the art. Briefly, a lateral flow device is an analytical device having as its essence a test strip, through which flows a test sample fluid that is suspected of containing an analyte of interest. The test fluid and any suspended analyte can flow along the strip to a detection zone in which the analyte (if present) interacts with a capture agent and a detection agent to indicate a presence, absence and/or quantity of the analyte.

Numerous lateral flow analytical devices have been disclosed, and include those shown in U.S. Pat. Nos. 4,313,734; 4,435,504; 4,775,636; 4,703,017; 4,740,468; 4,806,311; 4,806,312; 4,861,711; 4,855,240; 4,857,453; 4,943,522; 4,945,042; 4,496,654; 5,001,049; 5,075,078; 5,126,241; 5,451,504; 5,424,193; 5,712,172; 6,555,390; 6,258,548; 6,699,722; 6,368,876 and 7,517,699, each of which is incorporated by reference.

Many lateral flow devices are one-step lateral flow assays in which a biological fluid is placed in a sample area on a bibulous strip (though non-bibulous materials can be used, and rendered bibulous, e.g., by applying a surfactant to the material), and allowed to migrate along the strip until the liquid comes into contact with a specific binding partner (such as an antibody) that interacts with an analyte (such as one or more proteins) in the liquid. Once the analyte interacts with the binding partner, a signal (such as a fluorescent or otherwise visible dye) indicates that the interaction has occurred. Multiple discrete binding partners (such as antibodies) can be placed on the strip (for example in parallel lines) to detect multiple analytes (such as two or more proteins) in the liquid. The test strips can also incorporate control indicators, which provide a signal that the test has adequately been performed, even if a positive signal indicating the presence (or absence) of an analyte is not seen on the strip.

Lateral flow devices have a wide variety of physical formats that are equally well known in the art. Any physical format that supports and/or houses the basic components of a lateral flow device in the proper function relationship is contemplated by this disclosure.

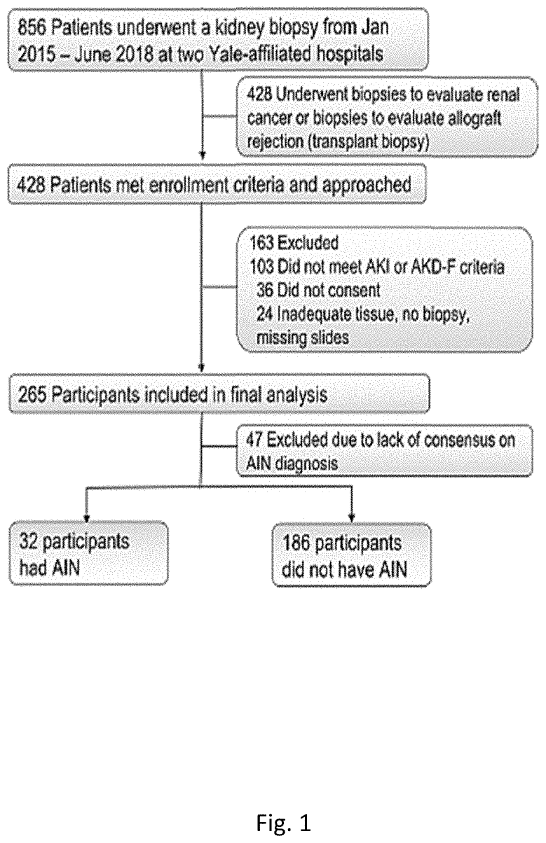

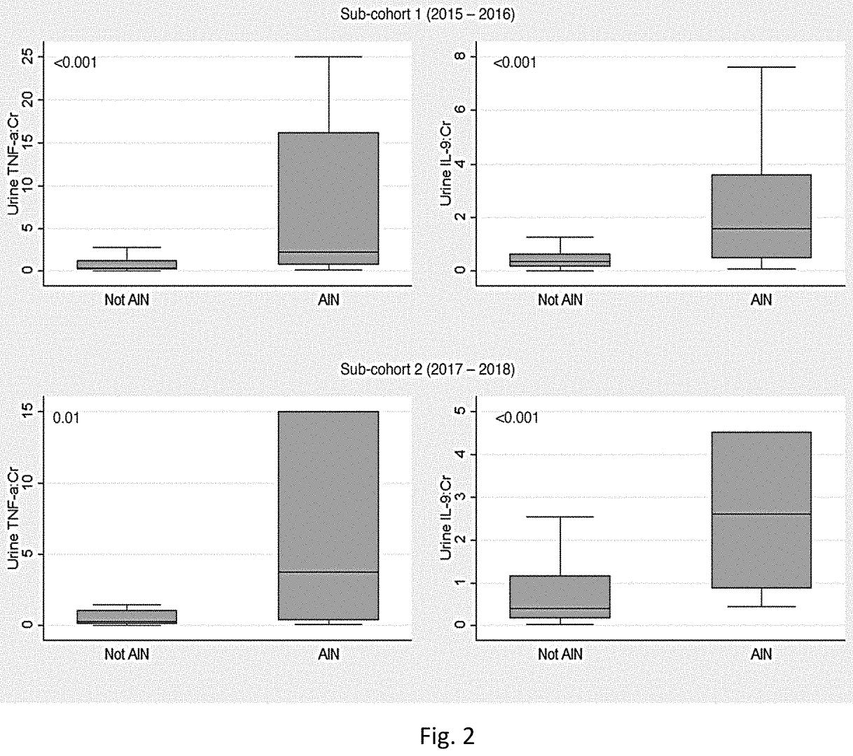

The basic components of a particular embodiment of a lateral flow device are illustrated in FIGS. 1 and 2 which comprise a sample pad, a conjugate pad, a migration membrane, and an absorbent pad.

The sample pad (such as the sample pad in FIGS. 1 and 2) is a component of a lateral flow device that initially receives the sample, and may serve to remove particulates from the sample. Among the various materials that may be used to construct a sample pad (such as glass fiber, woven fibers, screen, non-woven fibers, cellosic fibers or paper) or a cellulose sample pad may be beneficial if a large bed volume is a factor in a particular application. Sample pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

With respect to the migration membrane, the types of membranes useful in a lateral flow device include but are not limited to nitrocellulose (including pure nitrocellulose and modified nitrocellulose) and nitrocellulose direct cast on polyester support, polyvinylidene fluoride, or nylon).

The conjugate pad (such as conjugate pad in FIGS. 1 and 2) serves to, among other things, hold a detector reagent. Suitable materials for the conjugate pad include glass fiber, polyester, paper, or surface modified polypropylene.

Detector reagent(s) contained in a conjugate pad is typically released into solution upon application of the test sample. A conjugate pad may be treated with various substances to influence release of the detector reagent into solution. For example, the conjugate pad may be treated with PVA or PVP (0.5% to 2%) and/or Triton X-100 (0.5%). Other release agents include, without limitation, hydroxypropylmethyl cellulose, SDS, Brij and β-lactose. A mixture of two or more release agents may be used in any given application.

With respect to the absorbent pad, the pad acts to increase the total volume of sample that enters the device. This increased volume can be useful, for example, to wash away unbound analyte from the membrane. Any of a variety of materials is useful to prepare an absorbent pad, for example, cellulosic filters or paper. In some device embodiments, an absorbent pad can be paper (i.e., cellulosic fibers). One of skill in the art may select a paper absorbent pad on the basis of, for example, its thickness, compressibility, manufacturability, and uniformity of bed volume. The volume uptake of an absorbent made may be adjusted by changing the dimensions (usually the length) of an absorbent pad.

In operation of the particular embodiment of a lateral flow device, a fluid sample containing an analyte of interest, such as one or more proteins described herein, is applied to the sample pad. In some examples, the sample may be applied to the sample pad by dipping the end of the device containing the sample pad into the sample (such as urine) or by applying the sample directly onto the sample pad.

From the sample pad, the sample passes, for instance by capillary action, to the conjugate pad. In the conjugate pad, the analyte of interest, such as a protein of interest, may bind (or be bound by) a mobilized or mobilizable detector reagent, such as an antibody (such as antibody that recognizes one or more of the proteins described herein). For example, a protein analyte may bind to a labeled (e.g., gold-conjugated or colored latex particle-conjugated) antibody contained in the conjugate pad. The analyte complexed with the detector reagent may subsequently flow to the test line where the complex may further interact with an analyte-specific binding partner (such as an antibody that binds a particular protein, an anti-hapten antibody, or streptavidin), which is immobilized at the proximal test line. In some examples, a protein complexed with a detector reagent (such as gold-conjugated antibody) may further bind to unlabeled, oxidized antibodies immobilized at the proximal test line. The formation of a complex, which results from the accumulation of the label (e.g., gold or colored latex) in the localized region of the proximal test line, is detected. The control line may contain an immobilized, detector-reagent-specific binding partner, which can bind the detector reagent in the presence or absence of the analyte. Such binding at the control line indicates proper performance of the test, even in the absence of the analyte of interest.

In one embodiment, the control line detects the presence of one of IgG, IgD, IgA or another constituent of urine. In one embodiment, the control line detects the presence of one of glycoproteins, secretory IgA, lactoferrin, lysozyme and peroxidase, or another constituent of saliva.

The test results may be visualized directly, or may be measured using a reader (such as a scanner). The reader device may detect color, fluorescence, luminescence, radioactivity, or any other detectable marker derived from the labeled reagent from the readout area (for example, the test line and/or control line).

In another embodiment of a lateral flow device, there may be a second (or third, fourth, or more) test line located parallel or perpendicular (or in any other spatial relationship) to the test line in the test result. The operation of this particular embodiment is similar to that described elsewhere herein with the additional considerations that (i) a second detector reagent specific for a second analyte, such as another antibody, may also be contained in the conjugate pad, and (ii) the second test line will contain a second specific binding partner having affinity for a second analyte, such as a second protein in the sample. Similarly, if a third (or more) test line is included, the test line will contain a third (or more) specific binding partner having affinity for a third (or more) analyte.

In one embodiment, a comparison of the control line to the test line yields the test result from the diagnostic system of the invention. In some instances, a valid result occurs when the control line is detected at a higher intensity level than the test line. For example, a valid result occurs when the control line is at least 5% or more, for example, 10%, 20%, 30%, 40%, 50%, 60%, 70%, 80%, 90%, 100% or more darker than the test line. In some instances, a valid result occurs when the control line is at least 0.5 fold or more, for example, 1 fold, 2 fold, 3 fold, 4 fold, 5 fold, 6 fold, 7 fold, 8 fold, 9 fold, 10 fold or more darker than the test line.

Point of Care Diagnostic and Risk Assessment Systems

The system of the invention can be applied to a point-of-care scenario. U.S. Pat. Nos. 6,267,722, 6,394,952 and 6,867,051 disclose and describe systems for diagnosing and assessing certain medical risks, the contents of which are incorporated herein. The systems are designed for use on site at the point of care, where patients are examined and tested, as well as for operation remote from the site. The systems are designed to accept input in the form of patient data, including, but not limited to biochemical test data, physical test data, historical data and other such data, and to process and output information, such as data relating to a medical diagnosis or a disease risk indicator. The patient data may be contained within the system, such as medical records or history, or may be input as a signal or image from a medical test or procedure, for example, immunoassay test data, blood pressure reading, ultrasound, X-ray or MRI, or introduced in any other form. Specific test data can be digitized, processed and input into the medical diagnosis expert system, where it may be integrated with other patient information. The output from the system is a disease risk index or medical diagnosis.

Point of care testing refers to real time diagnostic testing that can be done in a rapid time frame so that the resulting test is performed faster than comparable tests that do not employ this system. For example, the exemplified immunoassay disclosed and described herein can be performed in significantly less time than the corresponding ELISA assay, e.g., in less than half an hour. In addition, point of care testing refers to testing that can be performed rapidly and on site, such as in a doctor's office, at a bedside, in a stat laboratory, emergency room or other such locales, particularly where rapid and accurate results are required.

In an exemplary embodiment, a point of care diagnostic and risk assessment system includes a reader for reading patient data, a test device designed to be read in the reader, and software for analysis of the data. A test strip device in a plastic housing is designed for use with the reader, optionally including a symbology, such as an alphanumeric character bar code or other machine-readable code, and software designed for analysis of the data generated from the test strip are also provided.

In one embodiment, a reader refers to an instrument for detecting and/or quantitating data, such as on test strips. The data may be visible to the naked eye, but does not need to be visible. Such readers are disclosed and described in the above-incorporated U.S. Pat. Nos. 6,267,722, 6,394,952 and 6,867,051. A reflectance reader refers to an instrument adapted to read a test strip using reflected light, including fluorescence, or electromagnetic radiation of any wavelength. Reflectance can be detected using a photodetector or other detector, such as charge coupled diodes (CCD). An exemplary reflectance reader includes a cassette slot adapted to receive a test-strip, light-emitting diodes, optical fibers, a sensing head, including means for positioning the sensing head along the test strip, a control circuit to read the photodetector output and control the on and off operation of the light-emitting diodes, a memory circuit for storing raw and/or processed data, and a photodetector, such as a silicon photodiode detector. It will be appreciated that a color change refers to a change in intensity or hue of color or may be the appearance of color where no color existed or the disappearance of color.

In one embodiment, a sample is applied to a diagnostic immunoassay test strip, and colored or dark bands are produced. The intensity of the color reflected by the colored label in the test region (or detection zone) of the test strip is, for concentration ranges of interest, directly proportional or otherwise correlated with an amount of analyte present in the sample being tested. The color intensity produced is read, in accordance with the present embodiment, using a reader device, for example, a reflectance reader, adapted to read the test strip. The intensity of the color reflected by the colored label in the test region (or detection zone) of the test strip is directly proportional to the amount of analyte present in the sample being tested. In other words, a darker colored line in the test region indicates a greater amount of analyte, whereas a lighter colored line in the test region indicates a smaller amount of analyte. The color intensity produced, i.e., the darkness or lightness of the colored line, is read using a reader device, for example, a reflectance reader, adapted to read the test strip.

A reflectance measurement obtained by the reader device is correlated to the presence and/or quantity of analyte present in the sample. The reader takes a plurality of readings along the strip, and obtains data that are used to generate results that are an indication of the presence and/or quantity of analyte present in the sample. The system may correlate such data with the presence of a disorder, condition or risk thereof.

As mentioned elsewhere herein, in addition to reading the test strip, the reader may (optionally) be adapted to read a symbology, such as a bar code, which is present on the test strip or housing and encodes information relating to the test strip device and/or test result and/or patient, and/or reagent or other desired information. Typically the associated information is stored in a remote computer database, but can be manually stored. Furthermore, the symbology can be imprinted when the device is used and the information encoded therein.

### Administration

In one embodiment, the systems as described elsewhere herein can be administered to patients taking a pharmaceutical associated with an increased risk of AIN, including, but not limited to, β-lactam antibiotics (e.g., penicillin and cephalexin), nonsteroidal anti-inflammatory drugs (e.g., celecoxib, diclofenac, diflunisal, etodolac, ibuprofen, indomethacin, ketoprofen, ketorolac, nabumetone, naproxen, oxaprozin, piroxicam, salsalate, sulindac, tolmetin), proton-pump inhibitors (eg. omeprazole, esomeprazole, lansoprazole, rabeprazole, pantoprazole, dexlansoprazole, etc.), anti-cancer immunotherapy agents (e.g., Ipilimumab, Nivolumab, Pembrolizumab, Atezolizumab, Avelumab, Duravlumab, etc.) as well as, rifampicin, sulfa medications, fluoroquinolones, diuretics, allopurinol, and phenytoin. In one embodiment, the systems as described elsewhere herein can be administered to patients taking a pharmaceutical associated with an increased risk of AIN for an extended period of time, e.g., for at least 10 consecutive days, at least 15 consecutive days, at least 20 consecutive days, at least 25 consecutive days, at least 1 month, at least 2 months, at least 3 months or for more than 3 months.

In one embodiment, the systems of the invention are administered to a patient by a provider in a clinical setting during a visit. In another embodiment, the systems are used by the patient outside of a clinical setting. In one embodiment, a patient using the system outside of the clinical setting could inform a physician of the results. In one embodiment, a patient using the system outside of the clinical setting could do so independent of reporting the results to a physician.

### Biological Samples

Biological samples to be analyzed using the invention may be of any biological tissue or fluid. Frequently the sample will be a “clinical sample” which is a sample derived from a patient. Typical samples for analysis include, but are not limited to, biological fluid samples such as sputum (a.k.a saliva), blood, plasma, milk, semen and urine.

Methods for collection of biological fluids from patients are well known in the art. In one embodiment, collection of a biological fluid for use in a lateral flow rapid visual test is with a sample cup or other receptacle. In one embodiment, a lateral flow device of the invention is inserted into a sample cup or other receptacle containing a biological fluid specimen. Receptacles appropriate for use in collecting biological fluid samples for use with the invention are not necessarily limited and are well known in the art. In one embodiment, a patient places an absorbent wick of a lateral flow device of the invention into their urine flow to collect the biological fluid for analysis. In one embodiment, a lateral flow device of the invention is inserted into an oral cavity and contacts the oral mucosa to collect the biological fluid for analysis.

In one embodiment, biological samples or aliquots of biological samples are shipped to a lab for analysis using a lab based test. In one embodiment, biological samples or aliquots of biological samples are frozen for shipment to a lab for analysis using a lab based test.

Test Results

In one embodiment, a lateral flow device provides results within 1 to 5 minutes. In this embodiment, the results can be read by the patient or provider and interpreted. In one embodiment, the patient sample is analyzed using a lab based test and results are sent by confidential electronic record or by confidential fax back to the patient or provider. Other methods of providing results to providers and patients are well known.

In one embodiment, the results are used by a provider to determine an appropriate course of treatment. In one embodiment, the test results are interpreted by a provider and used to inform a counseling strategy with the patient either in person or by phone, email, text message, or other communication medium. This includes but is not limited to a discussion with the patient, formulating a care plan, and altering a prescribed medication. Additionally, the provider can use this information to identify patients in need of a kidney biopsy (e.g., patients in which urine testing has shown that may be at increased risk of AIN including, but not limited to, patients with a urine TNF-α concentration >2 pg/mL, or patients with a urine IL-9 concentration >0.55 pg/mL.)

In one embodiment, the patient could use the system outside of a clinical setting. In one embodiment, the patient could use the system at the direction of a provider. In one embodiment, the patient could inform their provider of their results. This could include, but is not limited to, informing the provider after each individual test through a phone call, messaging, or digital app or performing multiple tests and providing the results to the provider at intermittent visits. In an alternative embodiment, the patient could use the system independently of provider oversight.

In one embodiment, testing can be performed at a frequency determined by a provider or research director. In one embodiment, testing can be performed daily, weekly, monthly, or at any appropriate frequency. For example, in one embodiment, testing can be performed before a pharmaceutical associated with increased risk of AIN is prescribed and subsequently prior to the prescription being approved for refill.

In one embodiment, a POCT of the invention can be used along with a handheld device. In one embodiment, a handheld device for use with a POCT of the invention analyzes the results of the POCT. In one embodiment, the analysis is performed using an electronic detection method incorporated into the handheld device. In one embodiment, the handheld device of the invention interfaces with a computer program. In one embodiment, a computer program is an application or web-based evaluation tool. In one embodiment, a user accesses a computer program to analyze, track, or visualize the test results. In one embodiment, a computer program for analyzing, tracking, or visualizing the test results from a POCT also serves to report test results to a physician or other party.

Controls with respect to the presence or absence of at least one biomarker associated with AIN or concentration of at least one biomarker associated with AIN may be markers abundant in at least one of urine, saliva, blood or plasma. As described elsewhere herein, comparison of the test patterns of the at least one biomarker associated with AIN to be tested with those of the controls can be used to identify the presence of the at least one biomarker associated with AIN. In this context, the control or control group is used for purposes of establishing proper use and function of the systems and assay of the invention. Therefore, mere detection of a at least one biomarker associated with AIN of the invention without the requirement of comparison to a control group can be used to identify the presence of the at least one biomarker associated with AIN. In this manner, the system according to the present invention may be used for qualitative (yes/no answer); semi-quantitative (−/+/++/+++/++++) or quantitative answer.

The concentration level of at least one biomarker associated with AIN in urine serves as a signpost for the increased risk of developing AIN. For example, a urine TNF-α concentration >2 pg/mL may indicate that a patient is at high risk of developing AIN, whereas a urine TNF-α concentration between 0.25 to 2 pg/mL may indicate that a patient is at some risk of developing AIN and a urine TNF-α concentration below 0.25 pg/mL may indicate that a patient is at low risk of developing AIN.

### Methods of Treatment

In one embodiment, a person diagnosed with AIN may be prescribed a pharmaceutical for treatment of AIN. In one embodiment, a pharmaceutical for treatment of AIN results in a decreased level of one or more biomarkers associated with AIN in a sample of a subject. In one embodiment, a pharmaceutical for treatment of AIN is an immunosuppressive agent. In various embodiments, immunosuppressive agents that may be administered to a subject diagnosed as having or at risk of developing AIN include, but are not limited to, calcineurin inhibitors (e.g., tacrolimus and cyclosporine), antiproliferative agents (e.g., mycophenolate mofetil, mycophenolate sodium, leflunomide and azathioprine), mTOR inhibitors (e.g., sirolimus and everolimus), corticosteroids (e.g., prednisone, budesonide, and prednisolone), biologics (e.g., abatacept, adalimumab, anakinra, certolizumab, etanercept, golimumab, infliximab, ixekizumab, natalizumab, secukinumab, tacilizumab, ustekinumab, and vedolizumab) and monoclonal antibodies (e.g., basiliximab, daclizumab, and muromonab).

In one embodiment, a person diagnosed with AIN or diagnosed as having an increased risk of AIN may be given an alternative treatment regimen or a drug holiday from a prescribed pharmaceutical agent. In one embodiment, a drug holiday is a period of at least 1 day, at least 2 days, at least 3 days, at least 4 days, at least 5 days, at least 6 days, at least 1 week, at least 2 weeks, at least 3 weeks, at least 4 weeks, at least 1 month or for more than 1 month. In one embodiment, at least one biomarker associated with AIN is measured before and after a drug holiday to determine if a pharmaceutical is associated with increased risk of AIN in the subject.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Inflammatory Mediators for Diagnosis of Biomarkers for Acute Interstitial Nephritis

Without being bound by theory, it was hypothesized that AIN is a delayed hypersensitivity reaction to drugs, which is mediated by a specific type of CD4+Th cells acting through release of characteristic cytokines, such as IFN-γ and IL-2 (type 1); IL-4, IL-5, and IL-13 (type 2); or IL-9 (type 9). Th1 and Th2 cells mediate drug-related delayed hypersensitivity reactions in other organs such as skin and lungs through release of their characteristic inflammatory mediators (Palm et al., 2012, Nature, 484(7395):465-472; Licona-Limon et al., 2013, Nat Immunol, 14(6):536-542). This hypothesis is supported by several findings in AIN. First, Th-cells account for the largest fraction of immune cells in the kidney biopsies from AIN patients and “tubulitis”, the phenomenon where immune cells cross from interstitium into the tubular space, is also caused by these Th-cells (D'Agati et al., 2989, Mod Pathol, 2(4):390-396; Spanou et al., 2006, J Am Soc Nephrol, 17(10):2919-2927). Second, drug-specific Th1/Th2 cells were isolated from the blood and kidneys of patients with AIN, and these cells produced the characteristic Th1/Th2 inflammatory mediators (Spanou et al., 2006, J Am Soc Nephrol, 17(10):2919-2927). Without being bound by theory, it was predicted that inflammatory mediators produced by Th1/Th2 cells, tumor necrosis factor (TNF)-α and IL-9, will be higher in patients with AIN as compared with others.

The Methods are Now Described

Study Design and Participants.

Participants were prospectively enrolled who were scheduled to undergo a clinically indicated kidney biopsy at 2 Yale University-affiliated hospitals: Yale New Haven Hospital and St. Raphael's Hospital (both in New Haven, Conn., USA) from January 2015 to June 2018 (Moledina et al., 2018, Clin J Am Soc Nephrol, 13(11):1633-1640; Moledina et al., 2018, Kidney Int Rep, 3(2):412-416). All consecutive sampling adult participants who met the Kidney Disease Improving Global Outcomes AKD criteria (Kellum et al., 2012, Kidney Int, 2(suppl 1): 1-138) were included. AKD criteria include AKI and allow for a less abrupt loss of renal function over 3 months. The former criteria were selected based on a prior study that showed that although the AKD criteria include over 90% of participants with AIN on biopsy, the AKI criteria miss about half of all AIN cases (Chu et al., 2014, Clin J Am Soc Nephrol, 9(7):1175-1182). If no baseline serum creatinine (SCr) was available to assess AKD criteria, participants with SCr at biopsy of greater than or equal to 1.5 mg/dl were enrolled. Kidney transplant recipients were excluded because acute rejection cannot reliably be differentiated from AIN on histology. Participants who were undergoing a kidney biopsy to evaluate a renal malignancy were also excluded.

Establishing AIN Diagnosis.

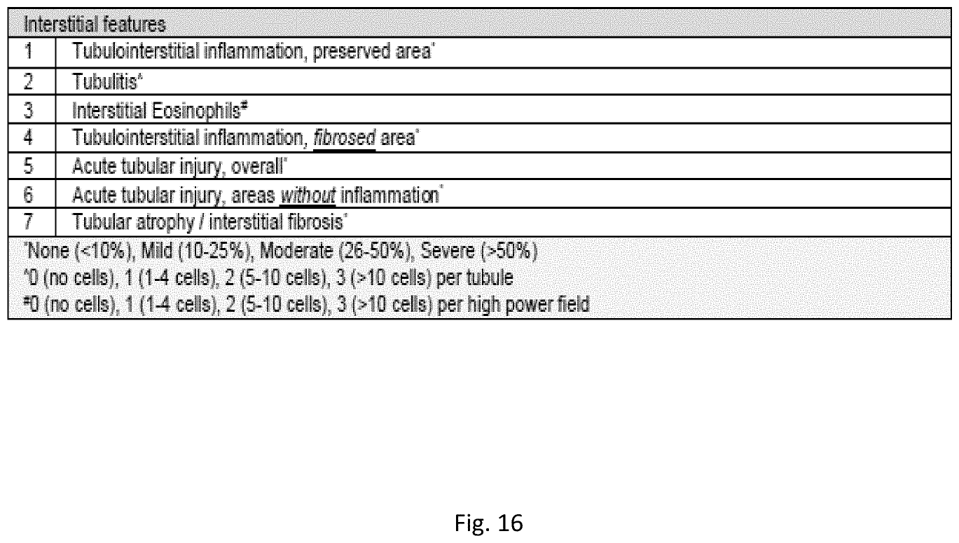

Three renal pathologists independently evaluated biopsy slides to establish AIN diagnosis. The pathologists were blinded to clinical history and official biopsy report. They evaluated all cases with official biopsy report of AIN (n=79) and a subset of those without any mention of AIN on the official biopsy report (n=28). These pathologists determined the presence or absence of AIN and rated the interstitial features on an ordinal scale developed for this study (FIG. 16). Out of 79 biopsies with official biopsy report of AIN, 32 (41%) were classified as AIN by all 3 pathologists, 23 (29%) were classified as AIN by 2 out of 3 pathologists, whereas 24 (30%) were classified as not AIN by at least 2 out of 3 pathologists (Table 2). None of the 28 biopsies without AIN on the official interpretation was classified as AIN by the adjudicating pathologists. A modest inter-rater agreement and κ statistic was noted among the pathologists for AIN diagnosis (agreement 63%-70%, Fleiss's κ=0.35). In the primary analysis, a biopsy was defined as “AIN” case when all 3 pathologists classified the biopsy as AIN and “not AIN” control when none reported AIN. Biopsies where one or 2 pathologists diagnosed AIN were excluded and all participants without official biopsy report of AIN were included as “not AIN” controls. In 3 sensitivity analyses, alternative case and control definitions were used. First, cases and controls were defined as the majority diagnosis among the pathologists without excluding any participant. Second, cases and controls were defined based on the diagnoses of the treating nephrologists after their review of the biopsies. Third, cases and controls were defined based on official biopsy interpretation.

Biomarker Testing

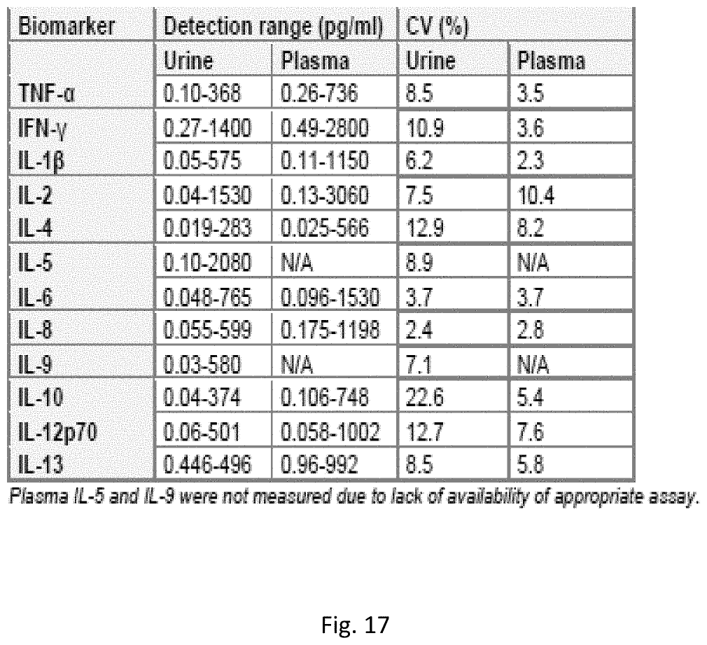

Biomarkers were measured from plasma and urine samples stored at −80° C. after a single controlled thaw. The sample processing protocol and biorepository tracking details have been described in a prior publication (Nadkarni et al., 2011, Clin Bioinforma, 1:22). Urine and plasma samples were collected a median (IQR) of 2.1 (−2.2 to 4.0) and 6.2 (1.6 to 26.7) hours before the biopsy. The manufacturer-validated 10-plex Proinflammatory Panel 1 from Mesoscale Discovery was used to test plasma TNF-α, IFN-γ, IL-1β, IL-2, IL-4, IL-6, IL-8, IL-10, IL-12p70, and IL-13. The above 10-plex panel was validated in the urine. A custom 2-plex urine assay for IL-5 and IL-9 was also created and validated. Mean interassay CV was 2.4% to 12% for all urine biomarkers except urine IL-10 (22.6%) and 2.3% to 10.4% for plasma biomarkers (FIG. 17). All urine biomarkers were normalized to urine creatinine to account for urine concentration differences. Urine albumin and creatinine measurements were performed using Randox RX Daytona machine and urine dipstick analysis using Clinitek Status analyzer (Siemens Healthcare Diagnostics Inc.). Urine sediment microscopy (Laxco LMC4BF, Fisher Scientific) was also performed and representative pictures were taken. The personnel measuring biomarkers, urine dipsticks, and urinalysis were blinded to the case status.

Sources of Data.

Demographic data, clinical history, laboratory results, medications, and nephrologists' pre- and postbiopsy diagnosis were collected through chart review of the Epic electronic health record (EHR) and cross-referenced with patient interviews. Scanned laboratory records were evaluated or physicians' offices were contacted if the above data were not available from the EHR. Biopsy-related complications were also assessed in a subset of participants enrolled until December 2017 (n=256); 12 (5%) participants required a blood transfusion, and 2 (0.8%) required an angiographic intervention because of biopsy-related bleeding (Moledina et al., 2018, Clin J Am Soc Nephrol, 13(11):1633-1640).

Immunofluorescence.

5 AIN and 15 non-AIN samples were selected for immunostaining for TNF-α and the mast cell marker FcεRI. Mast cells were used as a surrogate for IL-9 because IL-9 could not be reproducibly detected in human tissue via immunofluorescence or in situ hybridization. Formalin-fixed, paraffin-embedded human kidney was deparaffinized at 60° C. overnight followed by incubation in xylene for 20 minutes twice. Samples were rehydrated into tap water, and antigen retrieval was performed for 20 minutes at 96° C. in 1.25 mM EDTA, pH 8.0. Slides were cooled and blocked in TBS/0.05% Tween/0.3% BSA+1 μg/ml Fc block+25% heat-inactivated FBS (Fc block from BD Biosciences, 564220), for 2 hours at room temperature. Samples were then incubated with primary antibody overnight at 4° C. (TNF-α Abcam ab212899 at a concentration of 1:250 or FcεRI Abcam ab54411 at a concentration of 1:100). Samples were washed in TBS with Tween twice and TBS once and incubated with Alexa Fluor 546-conjugated goat anti-mouse at 1:100 for 1 hour at room temperature (Life Sciences A11003). Image quantification was performed using ×20 objective (Nikon Eclipse TE200) by an observer blinded to case status. Representative images were taken of AIN and non-AIN samples at identical exposure with ×40 objective. TNF-α and FcεRI were not co-stained because they were both mouse antibodies. Thus, to demonstrate colocalization, images from serial sections were manually aligned where morphology allowed, and when the same cell was captured on both sections, scoring for each marker was performed.

Statistics.

Data was presented as median (IQR) or count (percentage). Univariable comparison of biomarkers with AIN was performed using Kruskal-Wallis test after dividing the overall cohort into 2 temporal subcohorts (subcohort 1 from 2015 to 2017 and subcohort 2 from 2017 to 2018). TNF-α and IL-9 were selected for further analysis of biomarkers based on their association with AIN in both subcohorts. An alternative method of dividing the cohort by site of enrollment was also used. Sensitivity analyses were performed by using alternative case definitions of AIN as described above in Establishing AIN Diagnosis. The overall inter-rater agreement among pathologists (more than 2 raters providing more than 2 ratings) was calculated as described by Fleiss, Nee, and Landis using the “kap” command in Stata Statistical Software release 14 (StataCorp LP) (Fleiss et al., 1979, Psychological Bulletin, 86(5):974-977).

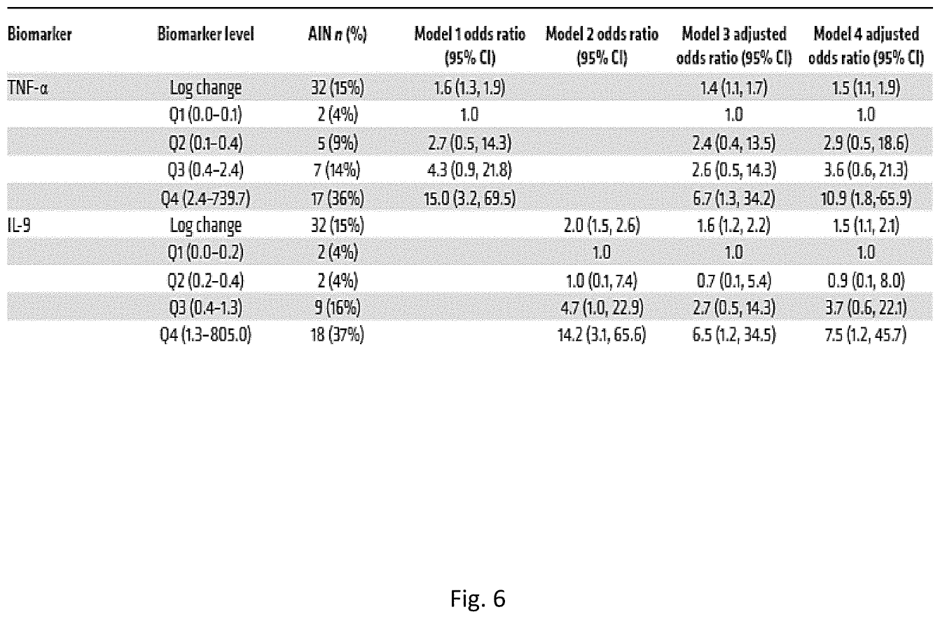

To test the independent association of these biomarkers with AIN, logistic models were fir with outcome of AIN and predictors as log-transformed biomarkers or quartiles of biomarkers. The analysis was controlled for blood eosinophils, urine protein, and urine leukocytes. To build a diagnostic model for AIN using currently available variables, variables thought to be associated with AIN (Perazella, 2014, Clin Nephrol, 81(6):381-388; Moledina and Perazella, 2016, J Nephrol, 29(5):611-616) were selected. The cohort was then divided into a random 70% subset, fit with a stepwise backward regression method with threshold for exclusion of P values greater than 0.2. This procedure was repeated 200 times, and variables that were selected in over 50% of the models were picked. For categorical variables, missing values were replaced with a separate term in analyses, and for continuous variables, the missing term was replaced with the median. To compare additional value of biomarkers over clinical information, 2 models were fit with outcome as AIN and predictors as clinicians' prebiopsy diagnosis (FIG. 6, model 3) and the clinical model developed as above (FIG. 6, model 4). The biomarkers were then added to these models and an increase in discrimination using change in the AUC was reported. Models were compared using the likelihood ratio test and tested model calibration using Hosmer-Lemeshow goodness-of-fit tests. To test the association of biomarkers with histological features, ordinal logistic models were fit with outcome as the interstitial feature reported by each individual pathologist and predictors as log-transformed biomarker values controlling for the pathologist and clustered at the level of the participant. The proportional odds assumption was tested in ordinal logistic regression using the Brant test. 2 biomarker cutoffs were tested to demonstrate clinical application. First, a high-specificity cutoff was tested, corresponding to the top 15% biomarker values in the cohort given the 15% prevalence of AIN. Second, a high-sensitivity cutoff was tested, corresponding to the median biomarker value in the cohort. Sensitivity and specificity at these cutoffs were reported. In addition, post-test probabilities of AIN at a range of pretest probabilities for each of these cutoffs are shown. To test the effect of corticosteroid therapy on biomarker levels, Wilcoxon's rank-sum test was used to compare biomarker values between those who did and did not receive steroids 7 days to 6 hours before the urine collection. Among those who received corticosteroid therapy before urine collection, linear regression was used to test the association of steroid dose with log-transformed biomarker values as well as values above and below the median. This analysis was controlled for postbiopsy diagnosis given the association of AIN with biomarkers. The calculations indicated that 36 AIN cases were required to detect a 50% difference in biomarker level between cases and controls (80% power, 2-sided α=0.05, assuming standard deviation=mean, and case/control=1:6). At least 33 cases were required to detect a 0.15 increase in AUC provided that the AUC of baseline model was 0.60 (Pencina et al., 2008, Stat Med, 27(2):157-172). Stata Statistical Software release 14 (StataCorp LP) was used for all analyses. All statistical tests were 2 sided with a significance level of P<0.05.

The Experimental Results are Now Described

Cohort Characteristics and Case Adjudication.

265 participants were enrolled who underwent a kidney biopsy for evaluation of acute kidney disease (AKD) between January 2015 and June 2018 at 2 Yale-affiliated hospitals (FIG. 1). Out of the 265 participants, 79 (30%) of biopsies were reported as AIN on official biopsy reports. Of these 79 biopsies, 32 (41%) were diagnosed as AIN by all 3 study pathologists and were included as cases in the primary analysis (Table 1). The 186 participants without AIN on official biopsy reports were included as controls. Baseline characteristics of study participants included in the primary analysis are presented in Table 1. At least 2 out of 3 pathologists diagnosed AIN in 55 participants, which were included as cases in a sensitivity analysis (Table 2).

TABLE 1
Baseline characteristics of participants who underwent kidney
biopsy for evaluation of acute kidney disease

| Characteristic | Overall (n = 218) | |
|---|---|---|
| Demographics and medical history | | |
| Age | 59 yr | (49-68 yr) |
| Female | 103 | (47%) |
| BMI | 29 kg/m2 | (25-34 kg/m2) |
| Black race | 55 | (25%) |
| Diabetes | 80 | (37%) |
| Hypertension | 164 | (75%) |
| Cirrhosis | 20 | (9%) |
| Chronic kidney disease | 149 | (73%) |
| Baseline laboratory features | | |
| Serum creatinine | 1.5 mg/dl | (1.1-2.1 mg/dl) |
| Estimated glomerular filtration rate | 41 ml/min | (26-62 ml/min) |
| Urine protein/creatinine ratio | 1.8 mg/mg | (0.6-4.6 mg/mg) |
| Features at biopsy | | |
| Located on floor | 121 | (56%) |
| Located in intensive care unit | 15 | (7%) |
| Outpatient | 82 | (38%) |
| Hospital 1 | 170 | (78%) |
| Acute kidney disease (excluding | 104 | (48%) |
| acute kidney injury) | | |
| Acute kidney injury, all cases | 114 | (52%) |
| Stage 1 acute kidney injury | 79 | (69%) |
| Stage 2 or higher acute kidney | 36 | (32%) |
| injury | | |
| Dialysis | 15 | (7%) |
| Urine output | 825 | (350-1435 ml/d) |
| Laboratory values at biopsy | | |
| Serum creatinine | 3.7 mg/dl | (2.3-5.2 mg/dl) |
| Blood urea nitrogen | 44 mg/dl | (31-64 mg/dl) |
| Hemoglobin level | 9.8 g/dl | (8.4-11.4 g/dl) |
| Platelets | 217A | (162A-276A) |
| Blood eosinophil count | 215/mm3 | (111/mm3 to 381/mm3) |
| Medication use | | |
| Proton pump inhibitor use | 89 | (41%) |
| Nonsteroidal antiinflammatory | 42 | (19%) |
| drug use | | |
| Antibiotic use | 112 | (52%) |
| Urine dipstick features | | |
| Leukocyte esterase, ≥2+ | 47 | (23%) |
| Blood, ≥2+ | 137 | (66%) |
| Protein, ≥2+ | 154 | (74%) |
| Urine microscopy features | | |
| White blood cell, at least 5/HPF | 39 | (19%) |
| White blood cell cast, at least | 5 | (2%) |
| 1/HPF | | |
| Granular cast, at least 1/HPF | 82 | (40%) |
| Red blood cells, at least 5/HPF | 61 | (42%) |
| Red blood cell cast, at least 1/HPF | 17 | (12%) |
| Dysmorphic red blood cells, at | 7 | (5%) |
| least 5/HPF | | |

Data are presented as median (IQR) or n (%). A1000 per mm³. HPF, high-power field.

TABLE 2
Participants selected for adjudication by three pathologists
and adjudication results

| Official | | | Number of pathologists | | |
|---|---|---|---|---|---|
| biopsy | | Selected for | diagnosing AIN | | |
| report | Total | adjudication | All 3 | 2 out of 3 | 1 |
| AIN | 79 | 79 (100%) | 32 (41%) | 23 (29%) | 24 (30%) |
| 1st | 38 | 38 | 21 (66%) | 10 (44%) | 7 (29%) |
| Diagnosis¹ | | | | | |
| 2_{nd} or 3_{rd} | 41 | 41 | 11 (34%) | 13 (57%) | 17 (71%) |
| Diagnosis | | | | | |
| Not AIN | 186 | 28 (15%) | 0 (0%) | 0 (0%) | 28 (100%) |

¹1st, 2nd, and 3rd diagnosis refer to the numerical order in which AIN was listed on the official biopsy report. Agreement on AIN diagnosis between pairs of pathologists ranged from 63-70% and overall Fleiss kappa was 0.35.

Urine TNF-α and IL-9 were Identified as Biomarkers of AIN.

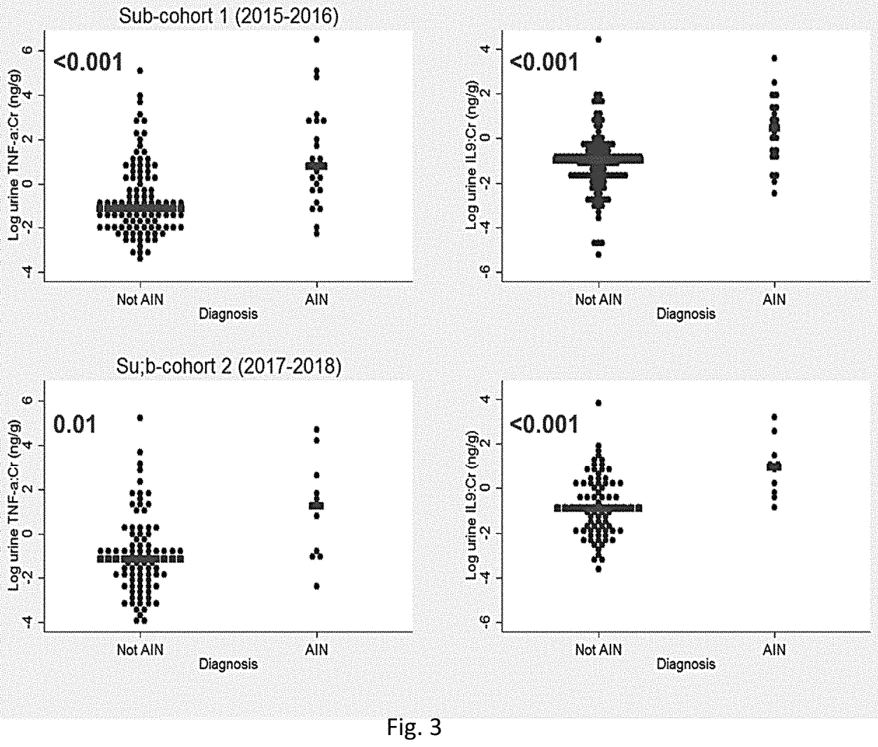

12 urine and 10 plasma inflammatory biomarkers were measured. These included cytokines specifically associated with CD4+ T cell subsets but also included more general inflammatory cytokines, such as TNF-α and IL-6. To identify biomarkers. For further analysis, the overall cohort was divided into 2 subcohorts separated by chronology of enrollment into 155 (59%) participants in cohort 1 (years 2015-2016) and 110 (41%) participants in cohort 2 (years 2017-2018). Out of the 22 total biomarkers tested, 3 urine biomarkers, TNF-α, IL-9, and IL-6, were associated with AIN in both subcohorts, whereas none of the plasma biomarkers was associated with AIN (Table 3). The 2 urine cytokines with the strongest association with AIN and biological plausibility, urine TNF-α and IL-9, were selected for further analysis (FIG. 2 and FIG. 3). TNF-α and IL-9 remained associated with AIN on an alternative validation technique where the cohort was divided by site of enrollment (Table 4). Consistent results were found in 3 sensitivity analyses evaluating association of urine TNF-α and IL-9 with alternative case definitions, including AIN diagnosed by at least 2 out of 3 study pathologists, AIN diagnosed by the treating clinicians after their review of the kidney biopsies, and AIN on official biopsy reports (Table 5).

TABLE 3
Comparison of urine and plasma biomarker levels between
AIN and controls in the two sub-cohorts of the study.

| | | | |
|---|---|---|---|
| Characteristic | AIN | NOT AIN | P |

Sub-cohort 1: January 2015-January 2017

| | | |
|---|---|---|
| N | 22 | 105 |

Urine

| | | | | | |
|---|---|---|---|---|---|
| TNF-α | 2.25 | (0.70, 16.13) | 0.33 | (0.14, 1.23) | 0.0001 |
| IL9 | 1.60 | (0.45, 3.60) | 0.38 | (0.17, 0.63) | 0.0002 |
| IL12p70 | 0.26 | (0.17, 0.34) | 0.12 | (0.07, 0.21) | 0.0004 |
| IL2 | 0.68 | (0.43, 2.72) | 0.32 | (0.20, 0.55) | 0.0005 |
| IL6 | 12.50 | (4.89, 48.15) | 2.64 | (1.02, 9.99) | 0.0006 |
| IL4 | 0.07 | (0.06, 0.13) | 0.05 | (0.02, 0.08) | 0.01 |
| IFN-γ | 1.63 | (0.36, 4.08) | 0.51 | (0.25, 0.85) | 0.01 |
| IL13 | 1.76 | (1.24, 3.02) | 1.00 | (0.57, 1.72) | 0.01 |
| IL1β | 2.97 | (1.59, 10.85) | 1.29 | (0.44, 3.68) | 0.02 |
| IL8 | 93.39 | (59.23, 177.22) | 50.63 | (16.09, 168.16) | 0.03 |
| IL5 | 0.12 | (0.08, 0.81) | 0.09 | (0.04, 0.22) | 0.05 |
| IL10 | 0.15 | (0.08, 0.24) | 0.09 | (0.04, 0.17) | 0.10 |

Plasma

| | | | | | |
|---|---|---|---|---|---|
| IL13 | 0.48 | (0.48, 0.48) | 0.48 | (0.48, 0.48) | 0.15 |
| IL8 | 9.06 | (4.15, 14.23) | 9.72 | (5.41, 17.70) | 0.26 |
| IL4 | 0.01 | (0.01, 0.04) | 0.01 | (0.01, 0.01) | 0.33 |
| IFN-γ | 6.61 | (3.33, 85.49) | 7.28 | (2.91, 17.37) | 0.35 |
| IL6 | 3.18 | (2.04, 19.69) | 3.52 | (1.53, 7.78) | 0.36 |
| IL2 | 0.13 | (0.06, 0.42) | 0.06 | (0.06, 0.30) | 0.42 |
| IL1β | 0.20 | (0.06, 0.33) | 0.15 | (0.06, 0.28) | 0.45 |
| IL10 | 0.47 | (0.27, 0.90) | 0.57 | (0.32, 1.32) | 0.50 |
| IL12p70 | 0.08 | (0.03, 0.13) | 0.08 | (0.03, 0.18) | 0.55 |
| TNF-α | 6.61 | (4.54, 10.07) | 6.31 | (4.04, 8.56) | 0.56 |

Sub-cohort 2: January 2017-June 2018

| | |
|---|---|
| 10 | 81 |

Urine

| | | | | | |
|---|---|---|---|---|---|
| TNF-α | 3.76 | (0.34, 15.03) | 0.32 | (0.10, 1.07) | 0.01 |
| IL9 | 2.61 | (0.86, 4.52) | 0.41 | (0.16, 1.16) | 0.001 |
| IL12p70 | 0.10 | (0.04, 0.22) | 0.08 | (0.04, 0.22) | 0.90 |
| IL2 | 0.29 | (0.14, 0.83) | 0.27 | (0.10, 0.52) | 0.41 |
| IL6 | 9.05 | (4.34, 15.44) | 3.14 | (1.27, 8.73) | 0.04 |
| IL4 | 0.03 | (0.03, 0.08) | 0.03 | (0.01, 0.06) | 0.44 |
| IFN-γ | 0.26 | (0.17, 5.34) | 0.59 | (0.26, 1.76) | 0.38 |
| IL13 | 1.06 | (0.23, 2.08) | 0.33 | (0.16, 1.33) | 0.26 |
| IL1β | 1.70 | (1.05, 7.99) | 0.62 | (0.36, 2.09) | 0.07 |
| IL8 | 94.18 | (28.11, 306.20) | 27.07 | (7.46, 129.75) | 0.07 |
| IL5 | 0.19 | (0.06, 0.54) | 0.10 | (0.06, 0.19) | 0.31 |
| IL10 | 0.23 | (0.15, 0.37) | 0.20 | (0.14, 0.35) | 0.56 |

Plasma

| | | | | | |
|---|---|---|---|---|---|
| IL13 | 0.19 | (0.19, 0.48) | 0.19 | (0.19, 0.55) | 0.81 |
| IL8 | 10.40 | (5.90, 14.28) | 10.13 | (5.53, 16.47) | 0.84 |
| IL4 | 0.03 | (0.02, 0.04) | 0.03 | (0.02, 0.04) | 0.63 |
| IFN-γ | 6.74 | (2.40, 14.84) | 3.18 | (1.56, 6.51) | 0.16 |
| IL6 | 3.30 | (1.85, 9.95) | 3.47 | (1.38, 8.33) | 0.68 |
| IL2 | 0.39 | (0.16, 0.67) | 0.22 | (0.13, 0.34) | 0.17 |
| IL1β | 0.30 | (0.18, 0.71) | 0.17 | (0.02, 0.35) | 0.11 |
| IL10 | 0.93 | (0.49, 1.42) | 0.52 | (0.32, 1.04) | 0.06 |
| IL12p70 | 0.10 | (0.08, 0.19) | 0.15 | (0.09, 0.24) | 0.29 |
| TNF-α | 9.52 | (7.46, 13.53) | 6.33 | (4.48, 9.88) | 0.01 |

TABLE 4
Alternate splitting of cohort to determine validity of biomarkers.

| Biomarker | AIN | Not AIN | P-value | AIN | Not AIN | P-value |
|---|---|---|---|---|---|---|
| | Year of enrollment | | | | | |
| | Sub-cohort 1: January 2015-January 2017 | | | Sub-cohort 2: January 2017-June 2018 | | |
| | 22 | 105 | | 1- | 81 | |
| TNF-α | 2.25 (0.70, 16.13) | 0.33 (0.14, 1.23) | <0.001 | 3.76 (0.34, 15.03) | 0.32 (0.10, 1.07) | 0.01 |
| IL9 | 1.60 (0.45, 3.60) | 0.38 (0.17, 0.63) | <0.001 | 2.61 (0.86, 4.52) | 0.41 (0.16, 1.16) | 0.001 |
| | Site of enrollment | | | | | |
| | Site 1 | | | Site 2 | | |
| | 24 | 146 | | 8 | 40 | |
| TNF-α | 2.25 (0.58, 0.58) | 0.30 (0.13, 0.83) | <0.001 | 3.97 (0.37, 11.15) | 0.41 (0.17, 2.80) | 0.04 |
| IL9 | 1.62 (0.55, 0.55) | 0.37 (0.16, 0.68) | <0.001 | 2.08 (0.55, 3.60) | 0.46 (0.22, 1.15) | 0.02 |

TABLE 5
Alternate approaches to acute interstitial nephritis diagnosis and biomarkers.

| | | | | | | |
|---|---|---|---|---|---|---|
| Biomarker | AIN | Not AIN | P-value | AIN | Not AIN | P-value |

| | |
|---|---|
| Phase 1: January 2015-January 2017 | Phase 2: January 2017-June 2018 |

A. Consensus diagnosis (Primary analysis)

| | 22 | 105 | | 1- | 81 | |
|---|---|---|---|---|---|---|
| TNF-α | 2.25 (0.70, 0.70) | 0.33 (0.14, 1.23) | <0.001 | 3.76 (0.34, 15.03) | 0.32 (0.10, 1.07) | 0.01 |
| IL9 | 1.60 (0.45, 0.45) | 0.38 (0.17, 0.63) | <0.001 | 2.61 (0.86, 4.52) | 0.41 (0.16, 1.16) | 0.001 |

B. Majority diagnosis (Sensitivity Analysis 1)

| | 35 | 120 | | 20 | 90 | |
|---|---|---|---|---|---|---|
| TNF-α | 1.45 (0.41, 0.41) | 0.34 (0.16, 1.35) | <0.001 | 2.37 (0.33, 11.37) | 0.37 (0.10, 1.18) | 0.01 |
| IL9 | 0.66 (0.29, 0.29) | 0.36 (0.17, 0.64) | 0.001 | 1.47 (0.26, 2.85) | 0.41 (0.16, 1.28) | 0.04 |

C. Clinician's post-biopsy diagnosis (Sensitivity Analysis 2)

| | 48 | 102 | | 31 | 76 | |
|---|---|---|---|---|---|---|
| TNF-α | 1.13 (0.38, 0.38) | 0.34 (0.15, 1.23) | <0.001 | 1.07 (0.14, 6.44) | 0.34 (0.11, 1.14) | 0.03 |
| IL9 | 0.59 (0.27, 0.27) | 0.34 (0.17, 0.60) | <0.001 | 1.00 (0.28, 2.96) | 0.41 (0.16, 1.27) | 0.03 |

D. AIN reported on official biopsy report (Sensitivity Analysis 3)

| | 50 | 105 | | 29 | 81 | |
|---|---|---|---|---|---|---|
| TNF-α | 1.16 (0.39, 0.39) | 0.33 (0.14, 1.23) | <0.001 | 2.37 (0.32, 13.45) | 0.32 (0.10, 1.07) | 0.008 |
| IL9 | 0.56 (0.22, 0.22) | 0.38 (0.17, 0.63) | 0.01 | 1.13 (0.26, 3.01) | 0.41 (0.16, 1.16) | 0.01 |

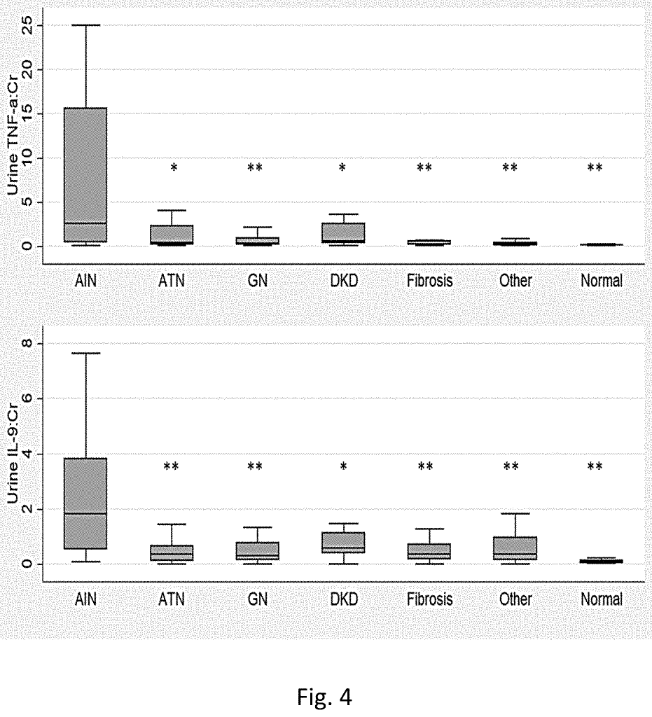

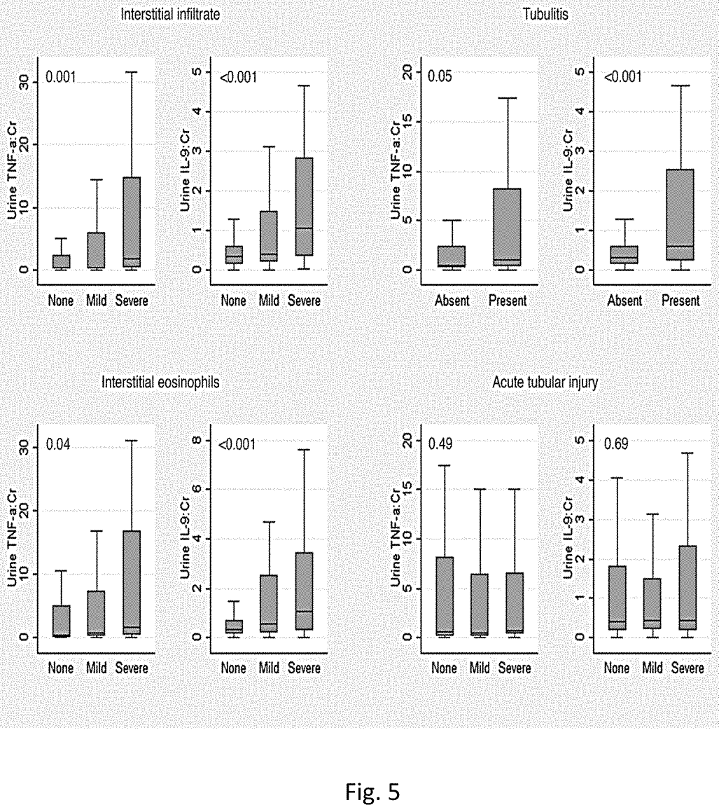

Participants with AIN also had higher urine TNF-α and IL-9 levels than those with other causes of AKD, including acute tubular injury, glomerular diseases, diabetic kidney disease, and progressive CKD (FIG. 4). These biomarkers were higher in AIN than in participants without any kidney disease. Urine TNF-α and IL-9 levels were also higher in those cases of AIN that were determined to be drug related (n=20, Table 6) than those without AIN, whereas levels were comparable between AIN cases thought to be drug related as compared with AIN due to other causes (Table 7). In addition, urine TNF-α and IL-9 were higher with increasing severity of interstitial histological features pathognomic of AIN, such as fraction of kidney tissue with lymphocytic infiltrate, presence of tubulitis, and number of interstitial eosinophils per high-power field (FIG. 5). In contrast, biomarkers did not correlate with degree of tubular injury reported on the adjudicated biopsies, which is the hallmark finding of acute tubular injury (ATI).

TABLE 6
Drug-induced acute interstitial nephritis and all controls

| Biomarker | AIN | Not AIN | P-value |
|---|---|---|---|
| N | 20 | 186 | |
| TNF-α | 3.16 (0.34, | 0.32 (0.13, 1.18) | 0.001 |
| | 43.61) | | |
| IL9 | 1.85 (0.66, | 0.39 (0.17, 0.78) | <0.001 |
| | 6.07) | | |

Drug induced AIN was thought to be due to antibiotics (n = 6), proton pump inhibitors (n = 3), non-steroidal anti-inflammatory drugs (n = 2), cancer immunotherapy (n = 3), and others (n = 6).
Median (IQR) are shown.
Wilcoxon Ranksum test.

TABLE 7
Comparison of biomarkers between drug-related and other
causes of acute interstitial nephritis

| Biomarker | Drug-related | Other AIN | P-value |
|---|---|---|---|
| N | 20 | 12 | |
| TNF-α | 3.16 (0.34, 43.61) | 2.05 (0.87, 6.79) | 0.78 |
| IL9 | 1.85 (0.66, 6.07) | 1.78 (0.55, 2.85) | 0.61 |

Median (IQR) are shown.
Wilcoxon Ranksum test.

Urine TNF-α and IL-9 were Independently Associated with AIN.

FIG. 6 shows sequential models testing association of log-continuous biomarkers and quartiles of biomarkers with AIN. Both log-continuous and highest quartiles of each biomarker were associated with higher odds of AIN in univariable analyses (models 1 and 2). The model containing both biomarkers (model 3) had an area under receiver operating characteristic curve (AUC) of 0.79 (0.71, 0.88). In a model controlling for key confounders, such as blood eosinophil count, dipstick leukocyturia, and dipstick proteinuria (model 4), the highest quartiles of TNF-α and IL-9 were independently associated with 10.9-fold and 7.5-fold higher odds of AIN, respectively.

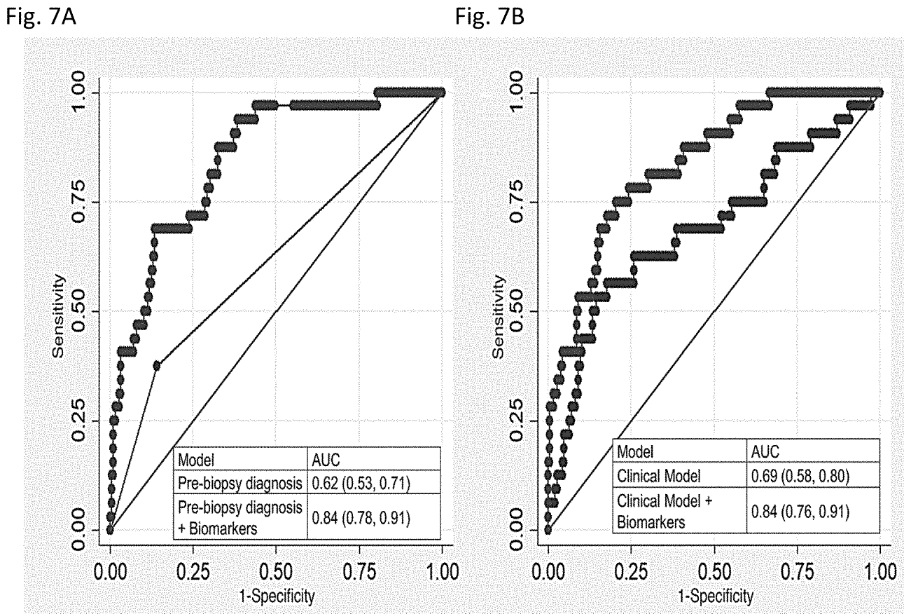

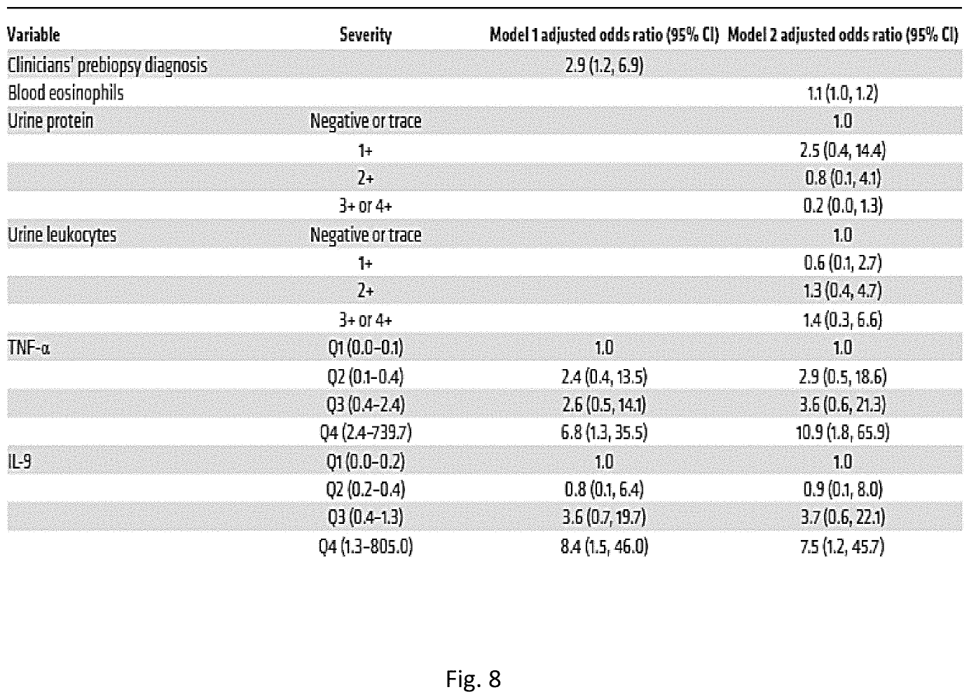

The contribution of biomarkers to 2 models was evaluated based on information currently available to clinicians. First, clinical charts were reviewed to determine whether AIN was the most likely diagnosis suspected by the clinical nephrologist before the biopsy, which had an AUC of 0.62 (0.53, 0.71) for AIN diagnosis. Second, a parsimonious model was created consisting of clinical variables typically associated with AIN. This model consisted of blood eosinophils, dipstick proteinuria, and dipstick leukocyturia and had an AUC of 0.69 (0.58, 0.80). Addition of biomarkers to either model improved the AUC significantly such that clinicians' prebiopsy diagnosis plus biomarkers had an AUC of 0.84 (0.78, 0.91, P<0.001) and the clinical model plus biomarkers had an AUC of 0.84 (0.76, 0.91, P<0.001) (FIG. 7). In the analysis containing biomarkers and clinical variables, the biomarkers were associated with AIN whereas the clinical variables were not (FIG. 8).

Clinical Application of Study Findings.

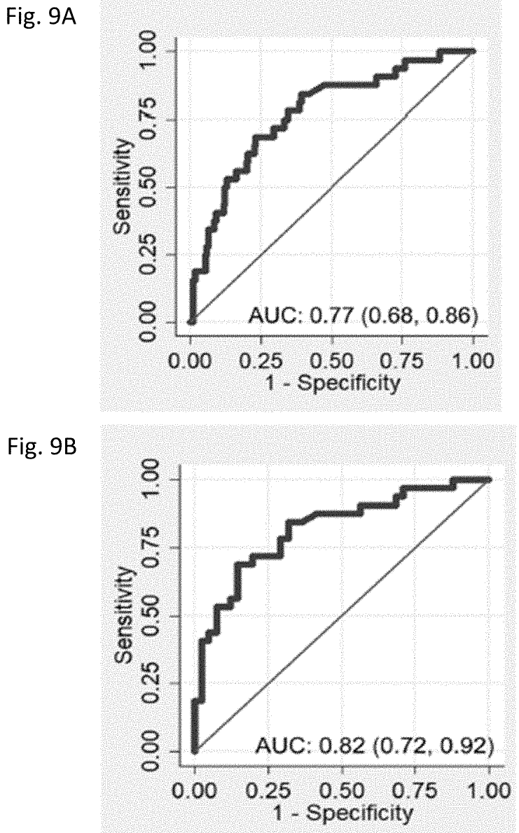

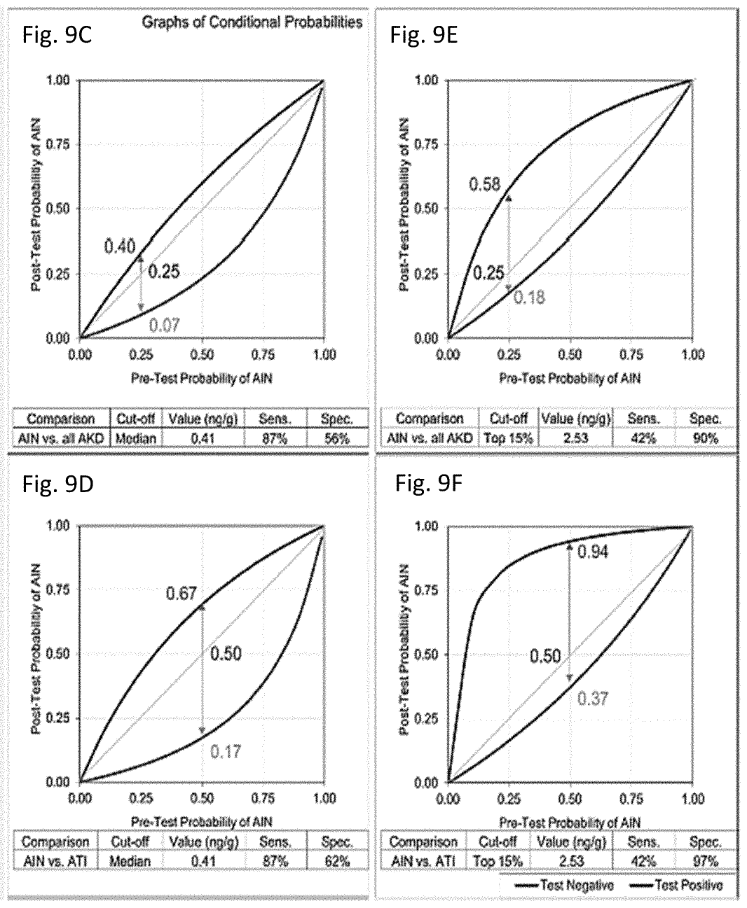

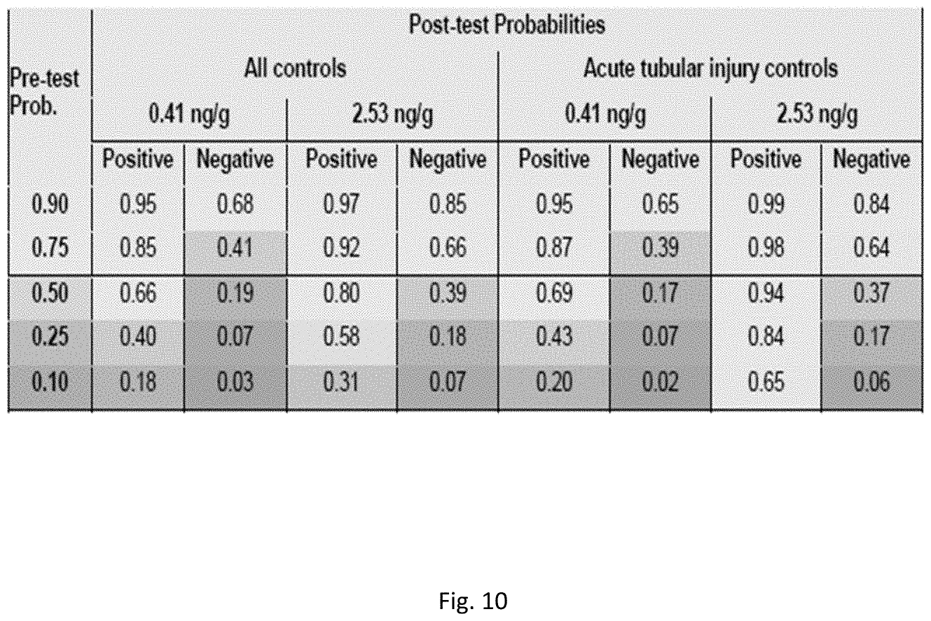

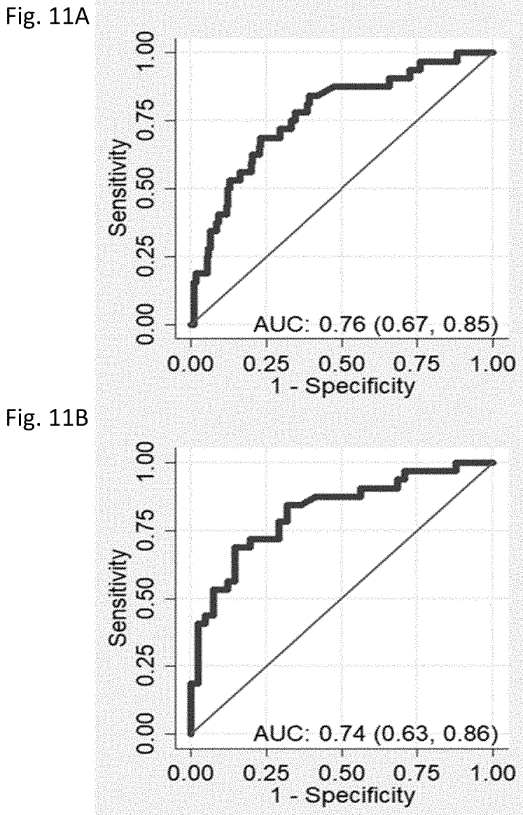

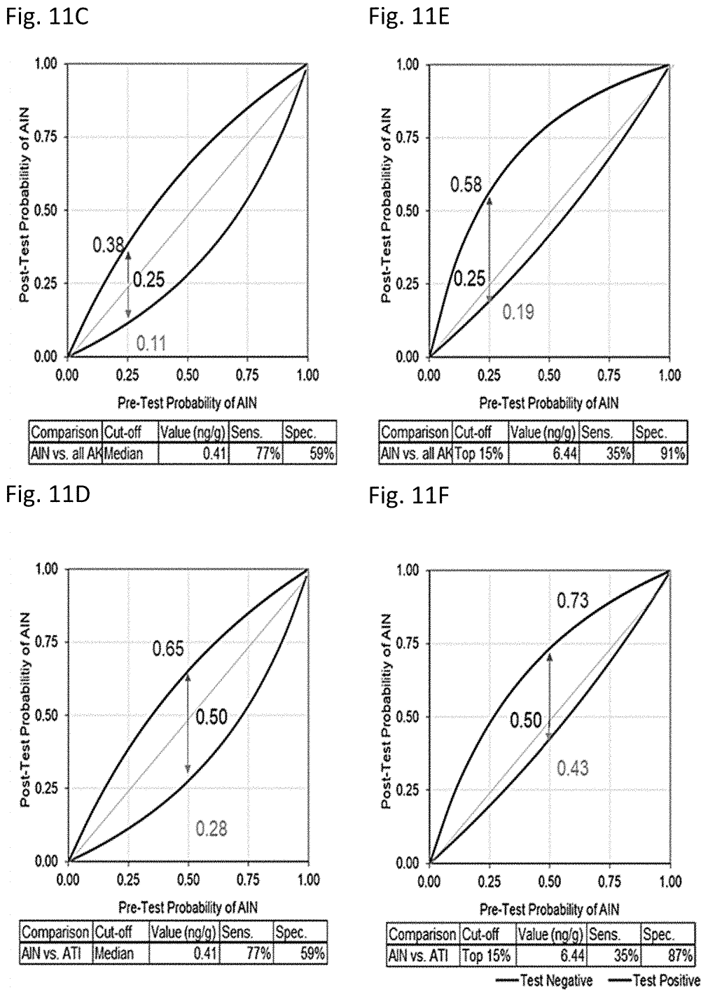

To demonstrate the clinical utility of urine IL-9 for clinical diagnosis of AIN, 2 cutoffs were evaluated: a high-specificity cutoff of 2.53 ng/g, which corresponds to the top 15% of study participants, and a high-sensitivity cutoff of 0.41 ng/g, which corresponds to the median biomarker value. FIG. 9A and FIG. 9B, shows AUC of urine IL-9 for AIN diagnosis when compared with AKD controls and ATI controls, respectively FIG. 9C through FIG. 9F, and FIG. 10 show post-test probabilities at a range of pretest probabilities at the 2 cutoffs. In a common scenario where a clinician wishes to distinguish AIN from ATI and has a pretest probability of 0.50 for AIN diagnosis, a positive IL-9 test at 2.53 ng/g cutoff will increase the post-test probability to 0.94, whereas a negative test at 0.41 cutoff will reduce post-test probability to 0.17. In both scenarios, the clinician may be able to avoid a kidney biopsy. Similar results were found for TNF-α (FIG. 11).

Determining the Source of TNF-α and IL-9.

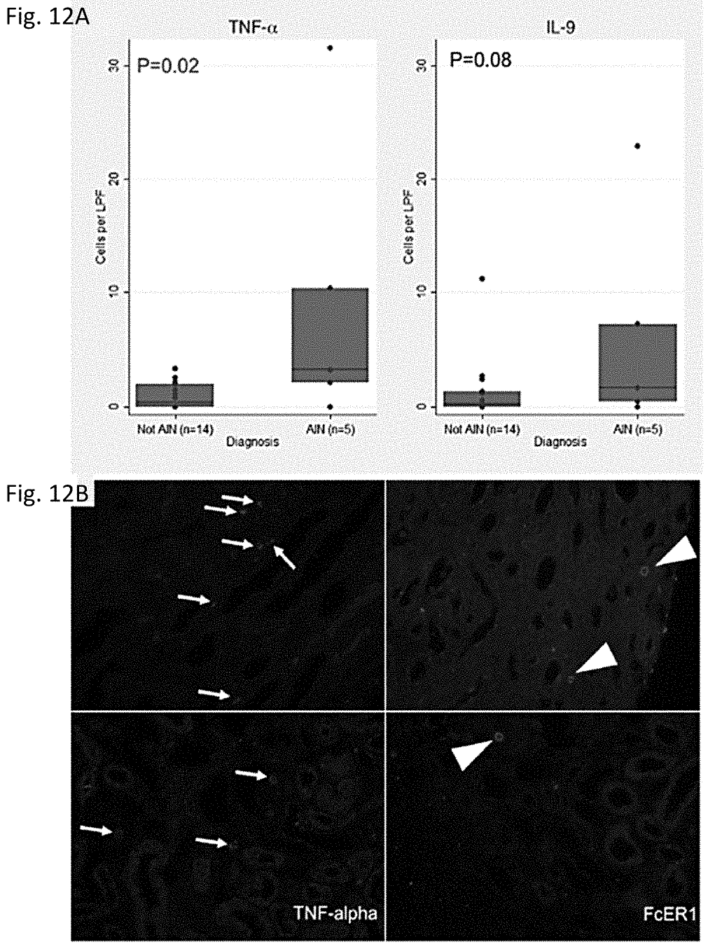

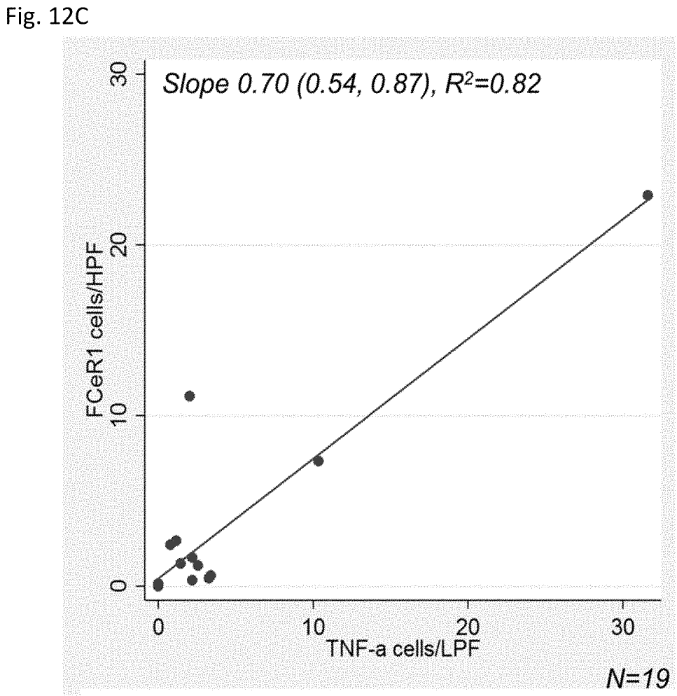

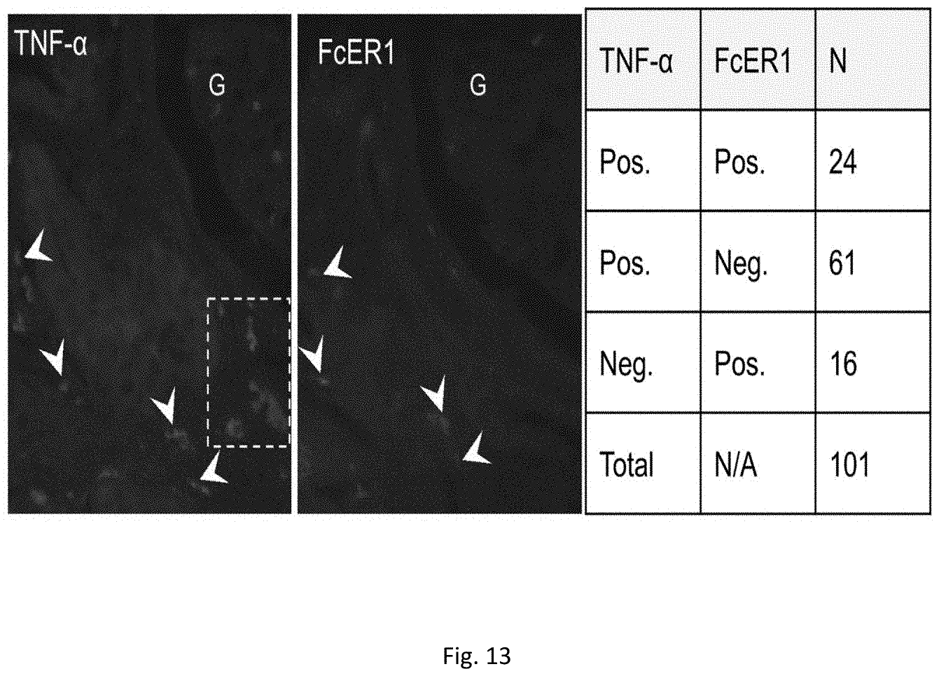

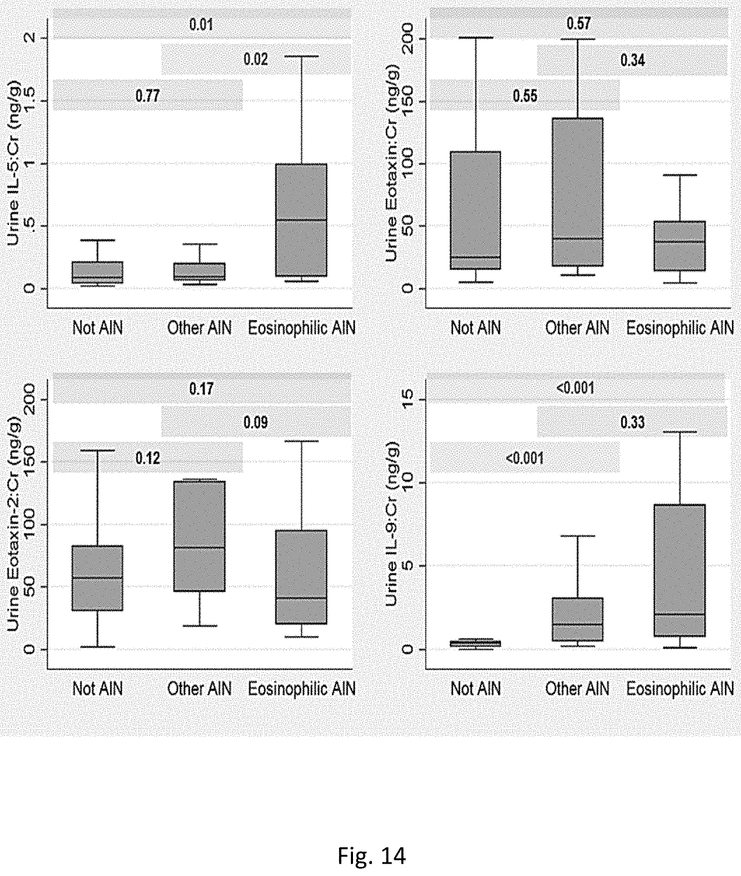

To determine whether the urine biomarkers were being produced in the kidneys or filtered from the blood, 3 approaches were used. First, kidney biopsies from study participants were used to identify intrarenal cells containing TNF-α and mast cells. Because kidney biopsies could not reliably be stained for IL-9, mast cells were stained, which are not normally present in kidneys and are considered downstream surrogates of IL-9 activity. FIG. 12 shows that biopsies with AIN had higher TNF-α+ cells than controls and shows a trend toward higher FcεRI+ cells, a mast cell marker. A high degree of correlation was noted between cells staining for TNF-α and FcεRI on the same biopsy (FIG. 12). It was also noted that there was a high degree of correlation between urine TNF-α and cells staining for TNF-α on kidney biopsy (rho=0.48, P=0.03) but not between urine IL-9 and FcεRI (rho=0.29, P=0.22) (Table 8). Among participants with AIN, 24 (28%) out of 85 TNF-α+ cells colocalized with FcεRI expression, and 24 (60%) out of FcεRI+ cells (n=40) colocalized with TNF-α expression (FIG. 13). Second, it was noted that although plasma biomarkers were not different between cases and controls, the ratio of urine to plasma TNF-α was higher in AIN than in controls (Table 9). Third, to determine whether the presence of biomarkers in urine was associated with abnormal glomerular filtration barrier, the ratio of urine biomarkers to urine albumin between AIN and controls was compared. It was found that this ratio was higher in AIN than in controls. Taken together, these approaches suggest that the urine biomarkers originated primarily in the kidneys. Eosinophils, IL-5, and AIN. Eosinophils in the renal tubulointerstitium are used histopathologically to diagnose AIN. AIN was diagnosed by the pathologists in all 16 (100%) biopsies with more than 5 eosinophils/high-power field (HPF), 12 (75%) biopsies with 1-5 eosinophils/HPF, and 4 (14%) biopsies with no eosinophils. Urine IL-5, an eosinophil-related cytokine, but not urine IL-9, was higher in AIN with more than 5 eosinophils/HPF than in AIN cases with less than or equal to 5 eosinophils/HPF (FIG. 14). However, an association of eotaxin-1 or eotaxin-2, 2 chemokines involved in eosinophil chemotaxis, with AIN was not identified.

TABLE 8
Correlation coefficients between cells on biopsy and urine biomarkers

| | TNF-α, cells | FcεRI, cells | Urine TNF-α | Urine IL-9 |
|---|---|---|---|---|
| TNF-α, cells | 1 | | | |
| FcεRI, cells | 0.77* | 1 | | |
| Urine TNF-α | 0.48* | 0.41 | 1 | |
| Urine IL-9 | 0.21 | 0.29 | 0.54* | 1 |

Spearman correlation coefficients (rho).
*indicates P < 0.05.
TNF, tumor necrosis factor;
IL, interleukin.

TABLE 9
Urine biomarker to albumin ratio and fractional excretion of biomarker

| Biomarker | AIN | Not AIN | P-value |
|---|---|---|---|
| N | 31 | 176 | |
| Urine TNF-α to | 0.24 (0.05, 0.55) | 0.04 (0.02, 0.10) | <0.001 |
| plasma TNF-α ratio | | | |
| Urine TNF-α to urine | 18.21 (2.34, 123.18) | 0.64 (0.18, 3.30) | <0.001 |
| albumin ratio | | | |
| Urine IL-9 to urine | 13.11 (1.27, 38.23) | 0.47 (0.17, 2.71) | <0.001 |
| albumin ratio | | | |

Wilcoxon Ranksum test.
Median (IQR) are shown.

Effect of Corticosteroid Therapy on Urine Biomarker Levels.

Corticosteroid therapy was administered to 35 (16%) study participants before urine was collected for biomarker measurement, which included 2 (6%) participants diagnosed as having AIN and 33 (18%) with other diagnoses. Urine TNF-α and IL-9 levels were comparable between those who received corticosteroids before urine collection compared with those who did not (Table 10). However, among those who received steroids, a higher corticosteroid dose was associated with lower urine IL-9 levels but not TNF-α levels in an analysis controlling for AIN diagnosis (model 2 in Table 11). It was noted that with each log increase in IL-9 levels, the corticosteroid dose administered was 180 mg (27 mg, 333 mg) lower. Similarly, those with IL-9 levels above the median had received a 330 mg (19 mg, 640 mg) to lower dose of corticosteroids.

TABLE 10
Comparison of urine biomarker levels between those who did and
did not receive corticosteroid therapy

| Biomarkers | Steroids before urine | No steroids | P-val. |
|---|---|---|---|
| | 35 | 183 | |
| TNF-a | 0.41 (0.16, 1.23) | 0.38 (0.14, 1.72) | 0.76 |
| IL-9 | 0.36 (0.18, 0.78) | 0.41 (0.17, 1.19) | 0.42 |

Wilcoxon rank sum test. Includes participants who received steroids between 7 days and 6 hours before urine collection.

TABLE 11
Association of urine biomarker levels with corticosteroid dose

| | | Steroid Dose in mg | Steroid Dose |
|---|---|---|---|
| | | (95% CI) | in mg (95% CI) |
| Biomarker | Comparison | Model 1 | Model 2 |
| TNF-a | Per log increase | −8 (−103, 87) | −19 (−120, 82) |
| | Below median | Ref. | Ref. |
| | Above median | −103 (−415, 208) | −131 (−453, 190) |
| IL-9 | Per log increase | −105 (−237, 28) | −180 (−333, −27) |
| | Below median | Ref. | Ref. |
| | Above median | −276 (−576, 24) | −330 (−640, −19) |

Linear regression analysis with biomarker quartile as outcome and steroid dose as predictor (Model 1). Model 2 controls for histological diagnosis (AIN yes/no).

The experiments presented demonstrate that urine TNF-α and IL-9 levels were consistently higher in participants with biopsy-proved, adjudicated AIN compared with other causes of AKD, whereas other plasma and urine biomarkers were comparable between the 2 groups. These biomarkers were higher in AIN than in various causes of AKD, including ATI, glomerular diseases, and diabetic kidney disease, as well as in participants without kidney disease. Urine TNF-α and IL-9 improved discrimination for AIN diagnosis as compared with the clinical nephrologist's prebiopsy diagnosis of AIN and a model consisting of currently available blood and urine tests. It is also demonstrated that there is an increase in cells staining for TNF-α and for FcεRI, a marker of mast cells, indicating that IL-9-driven mast cell release of TNF-α could be a potential source of this cytokine. Overall, these results indicate that concomitantly elevated levels of urine TNF-α and IL-9 are specific to AIN and may be a useful biomarker to distinguish AIN from other clinical causes of AKD.

Among the various causes of AKD, AIN is one of the few with a specific treatment. Yet, the clinical diagnosis of AIN is challenging because of its subacute presentation, lack of a pathognomonic clinical sign or symptom, and lack of a noninvasive diagnostic test. This challenge results in delay in diagnosis, increased fibrosis, and occurrence of CKD. For example, 1 study found that AIN was suspected in only 25% of cases from PPI before the biopsy (Muriithi et al., 2015, Kidney Int, 87(2):458-464). Unrecognized subclinical AIN is thought to be the cause of CKD in 2% to 3% of patients (Nochaiwong et al., 2018, Nephrol Dial Transplant, 33(2):331-342). Similar to earlier studies, it was found that the clinicians' prebiopsy diagnosis had an AUC of only 0.62 and a model with currently available clinical tests for AIN had an AUC of 0.69, which are indicative of the current challenges with the clinical diagnosis of AIN.

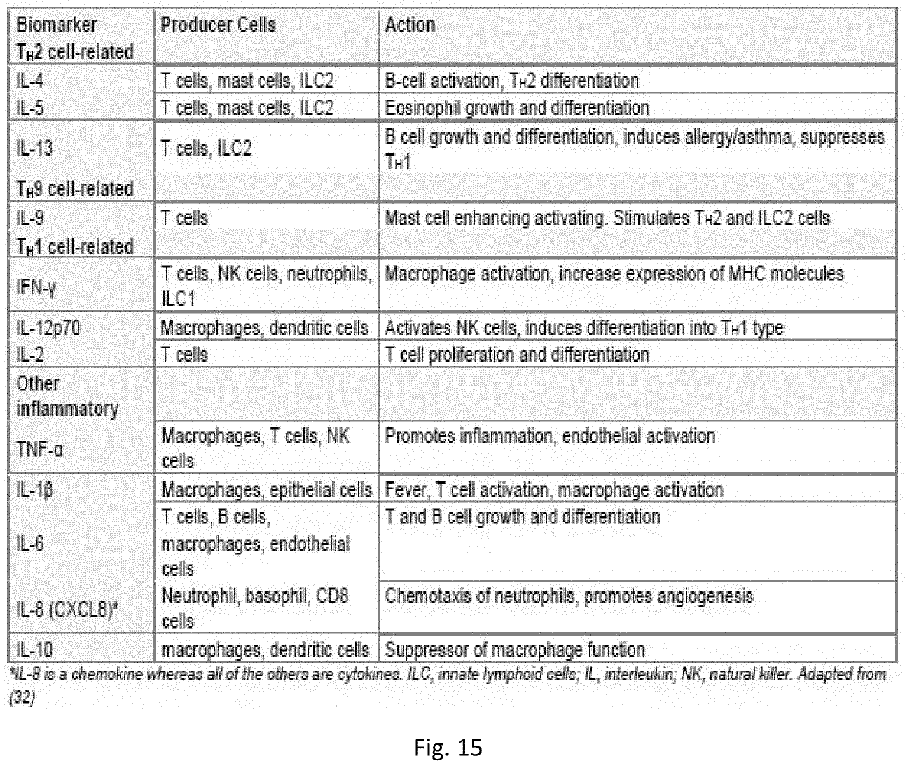

It was found that urine TNF-α and IL-9 had consistent association with AIN and significantly improved the discrimination for AIN diagnosis over the clinicians' prebiopsy impression and the model of clinical tests. Addition of these urine biomarkers to current clinical information could aid in the diagnosis of AIN by supplementing or replacing the kidney biopsy. Biomarkers were selected to be evaluated based on the hypothesis that AIN is a hypersensitivity reaction mediated by cytokines from specific T cell subsets and predicted that the relevant cytokines would be higher in AIN than other causes of AKD (FIG. 15). Kidney biopsies from patients with AIN are characterized by presence of lymphocytic infiltrate consisting predominantly of CD4+ T cells (D'Agati et al., 1989, Mod Pathol, 2(4):390-396), which produce both type 1 and type 2 cytokines (Spanou et al., 2006, J Am Soc Nephrol, 17(10):2919-2927). IL-9 was not evaluated in this earlier study. Type 2 immune responses, characterized by cytokines IL-4, IL-5, and IL-13, play an important role in allergen-induced diseases, including drug allergy. IL-5 is particularly associated with eosinophilic infiltrates. IL-9 is often associated with type 2 responses in allergic disorders, such as atopic dermatitis (Ciprandi et al., 2013, Pediatr Dermatol, 30(2):222-225), allergic asthma (Yao et al., 2013, Immunity, 38(2):360-372), and food allergy (Chen et al., 2015, Immunity, 43(4):788-802), and is produced by a distinct CD4+ T cell subset designated as Th9 (Ciprandi et al., 2013, Pediatr Dermatol, 30(2):222-225). Among these cytokines, it was found that IL-9 was most associated with AIN. IL-9 leads to differentiation, survival, and tissue accumulation of mast cells, including infiltration of mast cells in the renal tubulointerstitium (Godfraind et al., 1998, J Immunol, 160(8):3989-3996). Mast cells can also release preformed TNF-α and increase transcription of TNF-α (Gordon and Galli, 1990, Nature, 346(6281):274-276) and are a critical source of TNF-α in allergic diseases (Kim et al., 2007, Eur J Immunol, 37(4): 1107-1115). A study showed that AIN kidney biopsies had significantly higher mast cell numbers than biopsies with ATI (Zand et al., 2015, Clin Nephrol, 84(3):138-144). A trend toward higher mast cells was noted in AIN than other causes of AKD. It was also noted that a majority of FcεRI-staining mast cells colocalized with TNF-α. Thus, a unifying hypothesis based on the findings is that AIN is caused by IL-9-mediated activation of mast cells, which subsequently release TNF-α. Future studies could focus on further exploring the role of IL-9-producing CD4+ T cells and mast cells for understanding the pathogenesis of AIN. Moreover, although the current therapies in AIN provide nonspecific immunosuppression using corticosteroids, future studies could investigate therapies specific to TNF-α and mast cells in treatment of AIN.

Presence of eosinophils in the renal interstitium is suggestive of diagnosis of drug-induced AIN. In this study the pathologists were more likely to diagnose AIN if the biopsies had eosinophils in the renal interstitium. The key cytokine involved in eosinophil production, IL-5, and 2 chemokines involved in eosinophil chemotaxis in tissues, eotaxin-1 and eotaxin-2 were tested. Although IL-5 was not associated with AIN in the overall cohort, it was noted that the subset of AIN cases with more than 5 eosinophils/HPF had higher urine IL-5 than AIN cases with fewer eosinophils or non-AIN cases that. A recent study showed that despite the presence of eosinophils in the kidney tissue, urine eosinophils were neither sensitive nor specific for AIN (Muriithi et al., 2013, Clin J Am Soc Nephrol, 8(11):1857-1862). However, this study did not specifically study the subgroup with high tissue eosinophilia. It is often observed clinically that cases with antibiotic-induced AIN have many kidney tissue eosinophils whereas those related to nonsteroidal antiinflammatory drugs have few. Together these findings point to a subset of AIN cases with high degree of renal interstitial eosinophils and urine IL-5 levels. Anti-IL-5 therapies could be a potential treatment for this subgroup of AIN patients.

Among the type 1 cytokines tested in this study, none was higher in AIN, indicating that type 1 immune responses are not predominantly associated with inflammation in AIN. Moreover, all the significant differences in cytokine levels between AIN and controls were in the urine, whereas none was noted in the plasma. Inflammation in AIN is usually limited to the kidneys, which makes urine a likely source for detection of inflammatory mediators. Detection of cytokines in the plasma in renal-limited inflammation would require reabsorption into the circulation from the kidneys, where cytokine concentration would be diluted in the extracellular fluid volume. Moreover, controls in this study included patients with systemic vasculitis, sepsis, and other systemic illnesses, all of which are conditions that are expected to have increased plasma levels of cytokines. Thus, measuring urine, rather than plasma, cytokines provides specificity for renal inflammation in AIN.

This study design has several strengths. First, prospective enrollment allowed us to standardize sample collection, processing, storage, and biomarker measurement. Second, AIN diagnosis in our study was established by adjudication by 3 independent pathologists blinded to clinical history and biopsy report. Third, the consistency of these findings across various sensitivity analyses was tested using alternative definitions of AIN. Fourth, participants who were selected for a biopsy for evaluation of AKD by their nephrologists were selected as controls, ensuring generalizability to clinical practice. Finally, consistent association was shown across various subgroups to eliminate false positive associations.

In conclusion, the experiments presented herein demonstrate that urine TNF-α and IL-9 are consistently associated with AIN and improve discrimination over a clinician's prebiopsy diagnosis and a model of currently available clinical tests. These results could guide diagnostic approaches in patients suspected to have AIN for early management that could supplement or replace a kidney biopsy. Moreover, these findings point to potentially novel insights into the role of mast cells and Th9 cells in AIN for future mechanistic studies.

### Example 2: Urine 11-9 Levels Predict Response to Corticosteroid Therapy

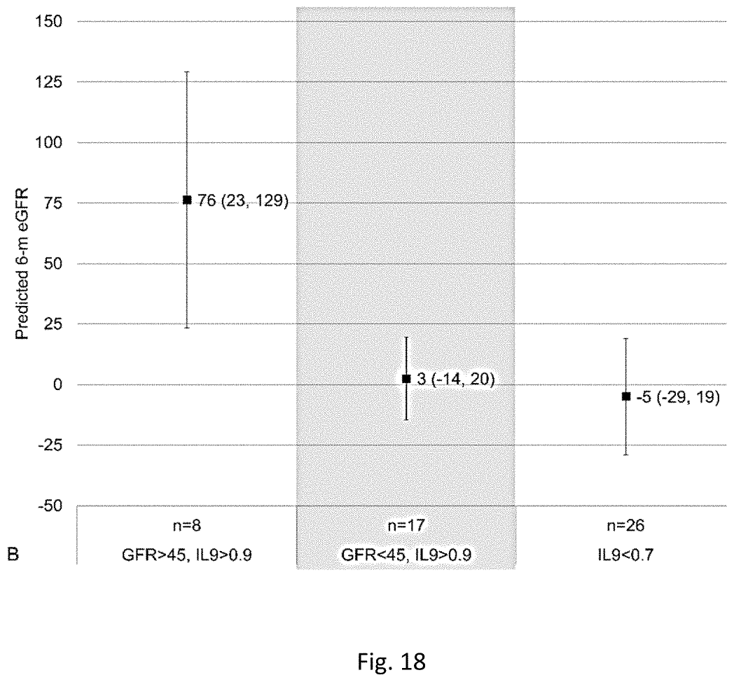

Management of patients with AIN often involves use of anti-inflammatory corticosteroid therapy in addition to discontinuation of the offending medication. However, corticosteroid therapy is associated with risks and may not be appropriate for all patients with AIN. Corticosteroid use was not effective at improving kidney function 6 months after AIN diagnosis when used in unselected patients (Table 12). However, in the subgroup of patients with higher severity of inflammation specifically those with higher urine IL-9 tended to have higher kidney function at follow-up with steroid use. Moreover, patients with high urine IL-9 (high inflammation) and high baseline kidney function had the best response to corticosteroid therapy (FIG. 18). These findings can help clinicians and researchers select the most appropriate patients to treat with corticosteroid or other immunosuppressive therapy in clinical practice and trials.

TABLE 12
Association of interleukin-9 with 6-month eGFR by steroid use

| Characteristic | Cut-off | 6-m eGFR (95% CI) | p-value | interact p-val |
|---|---|---|---|---|
| Steroid Use | . | 12.8 (−1.8, 27.4) | 0.08 | . |
| (overall) | | | | |
| IL-9, urine | | | . | 0.11 |
| | <0.66 | −6.2 (−31.5, 19.1) | 0.62 | . |
| | >0.90 | 18.6 (1.6, 35.6) | 0.03 | . |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A system for detecting at least one marker associated with acute interstitial nephritis (AIN) in a biological sample from a subject.

2. The system of claim 1, wherein the biological sample is at least one sample selected from the group comprising a urine sample, a saliva sample, a mucous sample, a whole blood sample, a blood plasma sample, a semen sample and a milk sample obtained from the subject.

3. The system of claim 1, wherein at least one marker is selected from the group consisting of a clinical marker and an inflammatory biomarker.

4. The system of claim 3, wherein at least one marker is selected from the group consisting of TNF-α, IL-9 and IL-5.

5. The use of the system of claim 1 for diagnosing an individual as having AIN or an increased risk of developing AIN.

6. A method of diagnosing a subject as having AIN or an increased risk of developing AIN, comprising:
a) detecting the level of at least one marker associated with AIN in a sample of the subject;
b) comparing the level of the at least one marker to the level of the marker in a comparator control, and
c) diagnosing the subject as having an increased risk of AIN based on detecting a significant difference between the level of the marker associated with AIN in the sample of the subject and the comparator control.

7. The method of claim 6, wherein the sample is at least one sample selected from the group comprising a urine sample, a saliva sample, a mucous sample, a whole blood sample, a blood plasma sample, a semen sample and a milk sample obtained from the subject.

8. The method of claim 6, wherein at least one marker is selected from the group consisting of a clinical marker and an inflammatory biomarker.

9. The method of claim 8, wherein at least one biomarker is selected from the group consisting of TNF-α, IL-9 and IL-5.

10. The method of claim 9, wherein risk of developing AIN is diagnosed when an increased level of at least one of TNF-α, IL-9 and IL-5 is detected as compared to a comparator control.

11. A method of diagnosing a subject as having AIN or an increased risk of developing AIN, comprising the steps of:
a) detecting the levels of at least two markers associated with AIN in at least one sample of a subject,
b) determining a health profile of the subject based on the levels of the at least two markers associated with AIN,
c) comparing the health profile of the subject to a diagnostic index generated from an analysis of AIN and non-AIN samples, and
d) diagnosing the subject as having an increased risk of AIN based on the diagnostic index.

12. The method of claim 11, wherein at least one sample is selected from the group comprising a urine sample, a saliva sample, a mucous sample, a whole blood sample, a blood plasma sample, a semen sample and a milk sample obtained from the subject.

13. The method of claim 11, wherein at least one marker is selected from the group consisting of a clinical marker and an inflammatory biomarker.

14. The method of claim 13, wherein at least one marker is selected from the group consisting of the level of blood eosinophils, the level of white blood cells in a urine sample, the level of hematuria, the level of albuminuria, the level of proteinuria, the baseline glomerular filtration rate, the level of TNF-α in a urine sample, the level of IL-5 in a urine sample, and the level of IL-9 in a urine sample.

15. A method of diagnosing and treating a subject as having AIN or an increased risk of developing AIN, comprising the steps of:
a) detecting the levels of at least two markers associated with AIN in at least one sample of a subject,
b) determining a health profile of the subject based on the levels of the at least two markers associated with AIN,
c) comparing the health profile of the subject to a diagnostic index generated from an analysis of AIN and non-AIN samples,
d) diagnosing the subject as having an increased risk of AIN based on the diagnostic index, and
e) administering a treatment regimen to the subject on the basis of the diagnosis.

16. The method of claim 15, wherein at least one sample is selected from the group comprising a urine sample, a saliva sample, a mucous sample, a whole blood sample, a blood plasma sample and a milk sample obtained from the subject.

17. The method of claim 15, wherein at least one marker is selected from the group consisting of a clinical marker and an inflammatory biomarker.

18. The method of claim 17, wherein at least one marker is selected from the group consisting of the level of blood eosinophils, the level of white blood cells in a urine sample, the level of hematuria, the level of albuminuria, the level of proteinuria, the baseline glomerular filtration rate, the level of TNF-α in a urine sample, the level of IL-5 in a urine sample, and the level of IL-9 in a urine sample.

19. The method of claim 15, wherein the treatment regimen is selected from the group consisting of, a drug holiday, a kidney biopsy, and an immunosuppressive agent.
